(19) United States
(12) Patent Application Publication
(10) Pub. No.: US 2010/0311815 A1
(43) Pub. Date: Dec. 9, 2010

(54) MIR-101 CANCER MARKERS

(75) Inventors: Arul M. Chinnaiyan, Plymouth, MI (US); Sooryanarayana Lnu, Ann Arbor, MI (US); Qi Cao, Ann Arbor, MI (US)

Correspondence Address: Casimir Jones, S.C., 2275 DEMING WAY, SUITE 310, MIDDLETON, WI, 53562, US

(73) Assignee: THE REGENTS OF THE UNIVERSITY OF MICHIGAN, Ann Arbor, MI (US)

(21) Appl. No.: 12/710,706

(22) Filed: Feb. 23, 2010

Related U.S. Application Data
(60) Provisional application No. 61/154,541, filed on Feb. 23, 2009.

(51) Int. Cl. A61K 31/7088; A61P 35/00; C12N 5/09

(52) U.S. Cl. 514/44 R; 435/325

(57) ABSTRACT

The present invention relates to compositions and methods for cancer diagnostics, including but not limited to, cancer markers. The present invention further provides novel markers useful for the diagnosis, characterization, and treatment of prostate cancers.

### CROSS REFERENCE TO RELATED APPLICATIONS

This application claims priority to provisional patent application 61/154,541, filed Feb. 23, 2009, which is herein incorporated by reference in its entirety.

### STATEMENT REGARDING FEDERALLY SPONSORED RESEARCH OR DEVELOPMENT

This invention was made with government support under grant numbers CA97063, CA69568, and 111274 awarded by the National Institutes of Health and grant number W81XWH-08-0110 awarded by the Army Medical Research and Material Command. The government has certain rights in the invention.

### FIELD OF THE INVENTION

The present invention relates to compositions and methods for cancer diagnostics, including but not limited to, cancer markers. The present invention further provides novel markers useful for the diagnosis, characterization, and treatment of prostate cancers.

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]; Maattanen et al, Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum. PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of new therapeutic targets and agents is needed.

### SUMMARY OF THE INVENTION

The present invention relates to compositions and methods for cancer diagnostics, including but not limited to, cancer markers. The present invention further provides novel markers useful for the diagnosis, characterization, and treatment of prostate cancers.

For example, in some embodiments, the present invention provides a method for identifying cancer in a patient comprising: detecting the presence or absence in the sample of miR-101, wherein the absence of miR-101 in the sample identifies cancer in the patient. In some embodiments, the cancer is, for example, prostate cancer, breast cancer, ovarian cancer, lung cancer gastric cancer, brain cancer, leukemia or colon cancer. In some embodiments, the detecting comprises detecting the presence or absence of miR-101 RNA. In some embodiments, the detecting comprises detecting the presence or absence of genomic DNA encoding miR-101. In some embodiments, the method further comprises the step of detecting the presence or absence of overexpression of EZH2 in the sample.

In some embodiments, the present invention provides a method of determining a treatment course of action, comprising detecting the presence or absence in the sample of miR-101; and determining a treatment course of action. In some embodiments, the treatment course of action is an EZH2 or miR-101 directed therapy.

Additional embodiments of the present invention provide a method of inhibiting the growth of a cancer cell or killing a cancer cell, comprising providing an exogenous nucleic acid encoding a micro RNA (e.g., miR-101, miR-203, or miR-200) to a cancer cell under conditions such that the growth of the cancer cell is inhibited or the cancer cell is killed. In some embodiments, the cancer is, for example, prostate cancer, breast cancer, ovarian cancer, lung cancer gastric cancer, brain cancer, leukemia or colon cancer. In some embodiments, the cancer cell is ex vivo, in vitro, or in an animal (e.g., a human or a non-human animal).

In some embodiments, the present invention provides a method of detecting metastatic prostate cancer, comprising determining a level of miR-203 in a prostate cancer sample from a subject; and detecting metastatic prostate cancer in the subject when the level of miR-203 is decreased relative to the level in subjects not diagnosed with metastatic prostate cancer.

Additional embodiments are described herein.

### DESCRIPTION OF THE FIGURES

FIG. 1 shows that miR-101 regulates EZH2 transcript and protein expression. (A) Venn diagram displaying miRNAs computationally predicted to target EZH2 from PicTar, miRanda, TargetScan, and MicroInspector. (B) Schematic of two predicted miR-101 binding sites in the EZH2 3′UTR. (C) miR-101 regulates EZH2 transcript expression. (D), miR-101 regulates Polycomb Group Complex 2 protein expression.

FIG. 2 shows the role of miR-101 in regulating cell proliferation, invasion and tumor growth. (A) miR-101 overexpression reduces cell proliferation. (B) miR-101 expression decreases cell invasion of DU145 prostate carcinoma cells. (C) AntagomiRs to miR-101 induce the invasiveness of benign immortalized H16N2 breast epithelial cells. (D) Overexpression of miR-101 attenuates prostate tumor growth.

FIG. 3 shows miR-101 regulation of the cancer epigenome through EZH2 and H3K27 tri-methylation. (A) Chromatin immunoprecipitation (ChIP) assay of the trimethyl H3K27 histone mark when miR-101 is overexpressed. (B) qRT-PCR of EZH2 target genes was performed using SKBr3 cells transfected with miR-101.

FIG. 4 shows genomic loss of the miR-101 in solid tumors. (A) miR-101 transcript levels are inversely correlated with EZH2 expression in prostate cancer progression. (B) Genomic PCR of miR-101-1 and miR-101-2 in prostate cancer progression. (C) Heatmap representation of matched normal, tumor, and metastatic samples (from right to left) in which miR-101 transcript, EZH2 transcript, and both miR-101-1 and miR-101-2 relative copy number were assessed. (D) Evidence that the miR-101-1 locus is somatically lost in tumors samples relative to matched normal samples.

FIGS. 5A-C show that overexpression of miR-101, but not miR-217 or control miRNA decreases the activity of the luciferase reporter encoding the 3′UTR of EZH2.

FIG. 6 shows that miR-101 binding sites were found in EED but not in SUZ12.

FIG. 7 shows inhibition of miR-101 expression in benign immortalized breast epithelial cells.

FIG. 8 shows that miR-101 overexpression in SKBr3 and DU145 cells attenuates cell proliferation.

FIG. 9 shows that miR-101 overexpression inhibits the in vitro invasive potential of SKBr3 breast cancer cells.

FIGS. 10A-B show that stable expression of miR-101 in DU145 cells showed a reduction in EZH2 expression and reduced invasion.

FIG. 11 shows that increased cell migration was inhibited by miR-101.

FIG. 12 shows that inhibition of miR-101 enhances neoplastic phenotype.

FIG. 13 shows that miR-101 inhibits anchorage independent growth

FIG. 14 shows that SKBr3 breast cancer and DU145 prostate cancer cells transfected with miR-101 or EZH2 siRNA for 7 days displayed a global decrease in tri-methyl H3K27 levels (FIG. 14A). The effect of miR-101 on H3K27 methylation was negated by overexpression of EZH2 (FIG. 14B).

FIG. 15 shows reduction in the tri-methyl H3K27 histone mark at the promoter of known PRC2 target genes such as ADRB2, DAB21P, CIITA and WNT1 in miR-101 overexpressing SKBr3 cells and EZH2 siRNA treated cells.

FIG. 16 shows that genes that were overexpressed at the 2-fold threshold were significantly overlapping in both the miR-101 and EZH2 siRNA transfected cells (P=6.08e-17).

FIG. 17 shows miR-101 expression in prostate cancer progression.

FIGS. 18A-B show that miR-101 has two genomic loci that are on chromosome 1 (miR-101-1) and chromosome 9 (miR-101-2).

FIGS. 19A-C show Agilent array CGH data for the region flanking the miR-101 from breast and gastric cancer.

FIGS. 20A-C show Agilent array CGH data for the region flanking the miR-101 from prostate cancer.

FIG. 21 shows loss of miR-101 in breast cancer.

FIG. 22 shows loss of the miR-101-2 locus in cancer.

FIG. 23 shows that miR-203 and 200 are EZH2 regulated.

FIGS. 24A-B show that EZH2 knock-down and miR-101 overexpression increase miR203 and 200 expression.

FIGS. 25A-B show that treatment with 5′Aza and SAHA increases miR-203 and 200 expression.

FIG. 26 shows that H3K27-me3 occupies the miR-203 region.

FIG. 27 shows that 5′Aza and SAHA inhibit H3K27me3 occupancy on miR-203 region.

FIG. 28 shows that miR-203 and 200 inhibit cell invasion.

FIG. 29 shows that miR-203 and 200 inhibit cell growth.

FIG. 30 shows that miR-203 inhibits anchorage-independent growth.

FIGS. 31A-C show that miR-203 inhibits sphere formation.

FIG. 32 shows that miR-203 inhibits tumor growth.

FIGS. 33A-C show microarray analysis of miR-203.

FIGS. 34A-B show that BMI1 is a target of miR203 and 200b,c.

FIG. 35 shows that RNF2 is a target of miR-200b and 200c.

FIG. 36 shows that miR-203 binds to BMI1 3′UTR.

FIGS. 37A-B show that miR-203 and 200 repress BMI1 and RNF2/RING2 protein levels.

FIG. 38 shows that antagomiR-203 increases BMI1 levels.

FIG. 39 shows that miR-203 and 200 repress BMI1 and RING2 expression.

FIG. 40 shows that miR-203 and 200 repress iPS factors.

FIG. 41 shows that miR-203 and 200 do not repress unrelated controls.

FIG. 42 shows that miR-203 inhibits H2A ubiquitination in stable cells.

FIGS. 43A-C show that miR-203 is downregulated in metastatic cancer.

FIG. 44 shows that EZH2, BMI1 and RING2 are upregulated in metastatic prostate cancer.

FIG. 45 shows that the miR-203 region is methylated in cancer.

### DEFINITIONS

To facilitate an understanding of the present invention, a number of terms and phrases are defined below:

As used herein, the term “gene upregulated in cancer” refers to a gene that is expressed (e.g., mRNA or protein expression) at a higher level in cancer (e.g., prostate cancer) relative to the level in other tissues. In some embodiments, genes upregulated in cancer are expressed at a level at least 10%, preferably at least 25%, even more preferably at least 50%, still more preferably at least 100%, yet more preferably at least 200%, and most preferably at least 300% higher than the level of expression in other tissues. In some embodiments, genes upregulated in prostate cancer are “androgen regulated genes.”

As used herein, the term “gene upregulated in prostate tissue” refers to a gene that is expressed (e.g., mRNA or protein expression) at a higher level in prostate tissue relative to the level in other tissue. In some embodiments, genes upregulated in prostate tissue are expressed at a level at least 10%, preferably at least 25%, even more preferably at least 50%, still more preferably at least 100%, yet more preferably at least 200%; and most preferably at least 300% higher than the level of expression in other tissues. In some embodiments, genes upregulated in prostate tissue are exclusively expressed in prostate tissue.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “cancer marker genes” refers to a gene whose expression level, alone or in combination with other genes, is correlated with cancer or prognosis of cancer. The correlation may relate to either an increased or decreased expression of the gene. For example, the expression of the gene may be indicative of cancer, or lack of expression of the gene may be correlated with poor prognosis in a cancer patient. In some embodiments, cancer marker genes serve as targets for anticancer therapeutics.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosome, the presence of membrane-bound organelles, and other characteristics commonly observed in eukaryotic organisms. Thus, the term includes, but is not limited to such organisms as fungi, protozoa, and animals (e.g., humans). As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products; such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for cancer diagnostics, including but not limited to, cancer markers. The present invention further provides novel markers useful for the diagnosis, characterization, and treatment of prostate cancers. MicroRNAs are regulatory, non-protein-coding, endogenous RNAs that have recently gained considerable attention in the scientific community. They are 18-24 nucleotides in length and are thought to regulate gene expression through translational repression by binding to a target mRNA (Lim et al., Science 2003; 299(5612):1540; Chen et al., Semin Immunol 2005; 17(2):155-65; Sevignani et al., Mamm Genome 2006; 17(3):189-202). They are also proposed to regulate gene expression by mRNA cleavage, and mRNA decay initiated by miRNA-guided rapid deadenylation (Wu et al., Proc Natl Acad Sci USA 2006; 103(11):4034-9). miRNAs are abundant, highly conserved molecules and predicted to regulate a large number of transcripts. To date the international miRNA Registry database has more than 600 human identified microRNAs (Griffiths-Jones et al., Nucleic Acids Res 2006; 34 (Database issue):D140-4) and their total number in humans has been predicted to be as high as 1,000 (Berezikov et al., Cell 2005; 120(1):21-4). Many of these microRNAs exhibit tissue-specific expression (Sood et al., Proc Natl Acad Sci USA 2006; 103(8):2746-51) and many are defined to be either tumor suppressors or oncogenes (Lee et al., Curr Opin Investig Drugs 2006; 7(6):560-4; Zhang et al., Dev Biol 2006; Calin et al., Nat Rev Cancer 2006; 6(11):857-66) and play a crucial role in variety of cellular processes such as cell cycle control, apoptosis, and haematopoiesis. Dysregulation of several miRNAs are thought to play a significant role in human disease processes including tumorigenesis (Hwang et al., Br J Cancer 2006; 94(6):776-80; Thomson et al., Genes Dev 2006; 20(16):2202-7).

Several microRNAs are located in the region of hot spots for chromosomal abnormalities (Calin et al., Oncogene 2006; 25(46):6202-10; Calin et al., Proc Natl Acad Sci USA 2004; 101(9):2999-3004). This results in abnormal expression of miRNAs which affect cellular functions. Recent studies indicate that selected miRNAs may play a role in human cancer pathogenesis. For example, deletions or mutations in genes that code for miRNA tumor suppressors lead to loss of a miRNA or miRNA cluster, and thereby contribute to oncogene deregulation (Zhang et al., supra; Calin et al., supra). The results of large-scale miRNA profiling studies using normal and cancer tissues show that a number of microRNAs are either overexpressed or downregulated in tumors (Alvarez-Garcia et al., Development 2005; 132(21):4653-62; Volinia et al., Proc Natl Acad Sci USA 2006; 103(7):2257-61; Cummins et al., Proc Natl Acad Sci USA 2006; 103(10):3687-92; Yanaihara et al., Cancer Cell 2006; 9(3):189-98; Iorio et al., Cancer Res 2005; 65(16):7065-70; Calin et al., Proc Natl Acad Sci USA 2004; 101(32):11755-60; Calin et al., N Engl J Med 2005; 353(17):1793-801; Pallante et al., Endocr Relat Cancer 2006; 13(2):497-508). It has been shown that miRNA genes are frequently located in cancer-associated genomic regions or fragile sites (Calin et al., Proc Natl Acad Sci USA 2004; 101(9):2999-3004). The genes encoding mir-15 and mir-16 are located at chromosome 13q14, a region that is deleted in the majority of B-cell chronic lymphocytic leukemias (B-CLL) indicating that mir-15 and mir-16 may function as tumor suppressors. let-7 miRNA family members are known to down regulate the oncogene RAS (Johnson et al., Cell 2005; 120(5):635-47). Its expression is reduced in tumors which in turn contributes to the elevated activity of the RAS pathway (Yanaihara et al., Cancer Cell 2006; 9(3):189-98). Expression levels of miR-143 and miR-145 were decreased in colon cancer tissues as well as in cancer cell lines (Michael et al., Mol Cancer Res 2003; 1(12):882-91). In contrast, several microRNAs are upregulated in cancer. Members of the miR-17 cluster provide an oncogenic function via their upregulated expression by c-Myc leading to effects on downstream genes which are mediators of cell cycle and apoptosis events (O'Donnell et al., Nature 2005; 435(7043):839-43).

Many microRNAs play a role during development and tissue differentiation (Pasquinelli et al., Curr Opin Genet Dev 2005; 15(2):200-5). miR-181, a microRNA that is strongly upregulated during differentiation, participates in establishing the muscle phenotype. Recent studies suggest that miR-181 down regulates the homeobox protein Hox-A11 (Naguibneva et al., Nat Cell Biol 2006; 8(3):278-84). Similarly miR-196 is involved in regulating HOXB8 confirming the significant roles played by microRNA during developmental processes. A recent study from Lim et al., (Yekta et al., Science 2004; 304(5670):594-6) showed that a few microRNAs can regulate large numbers of target mRNA and their studies also indicated that the miRNA can downregulate not only the proteins, but the transcript level of the target mRNA. Specific expression of microRNA are of prognostic significance, indicating that miRNAs are determinants of clinical aggressiveness (Volinia et al., supra, Iorio et al. Cancer Res 2005; 65(16):7065-70;Lu et al., Nature 2005; 435(7043):834-8). Thus, microRNA expression profiles can serve as a new class of cancer biomarkers. Breast cancer microRNA profiling studies by Iorio et al., (supra) indicated the expression patterns of several microRNAs were significantly different between normal and neoplastic tissues. This profiling study indicated miR-21 and miR-155 to be consistently up regulated and miR-10b, miR-125b and miR-145 to be down regulated. Further, breast tumor microRNA profiling distinguished normal from malignant breast tissue and correlated with breast cancer histopathologic features such as tumor size, nodal involvement, proliferative capacity and vascular invasiveness.

During experiments conducted during the course of development of embodiments of the present invention a search of the miRNA Registry database for microRNA that would target EZH2 implicated has miR-101. Further experiments conducted during the course of development of embodiments of the present invention demonstrated that EZH2 expression is inhibited by miR-101 and that loss of miR-101 leads to overexpression of EZH2 in cancer.

Accordingly, in some embodiments, the present invention provides methods of diagnosing cancer (e.g., prostate cancer) by detecting the loss of expression of miR-101, alone or in combination with overexpression of EZH2. In other embodiments, the present invention provides methods of treating cancer by replacing miR-101 expression (e.g., through gene therapy).

### I. Diagnostic Methods

The present invention provides DNA, RNA and protein based diagnostic methods that either directly or indirectly detect loss or decrease in expression of miR-101, alone or in combination with EZH2 overexpression. In some embodiments, methods detect miR-101 precursors. The present invention also provides compositions and kits for diagnostic purposes. Table 1 shows the sequence of miR-101 in different organisms.

TABLE 11

| | | | SEQ |
|---|---|---|---|
| | Accession | | ID |
| Organism | Number | Sequence | NO |
| Human | AF480499 | tacagtactg tgataactga ag | 1 |
| Human | AF480539 | ugcccuggcucaguuaucaca | 2 |
| Precursor | | gugcugaugcugucuauucua | |
| | | aagguacaguacugugauaac | |
| | | ugaaggauggca | |
| Mouse | AJ459730 | tacagtactg tgataactga | 3 |
| Bovine | DQ274888 | gtacagtact gtgataactg a | 4 |

The diagnostic methods of the present invention may be qualitative or quantitative. Quantitative diagnostic methods may be used, for example, to discriminate between indolent and aggressive cancers via a cutoff or threshold level. Where applicable, qualitative or quantitative diagnostic methods may also include amplification of target, signal or intermediary (e.g., a universal primer).

miR-101 loss or decrease in expression may be detected along with other markers in a multiplex or panel format. Markers are selected for their predictive value alone or in combination with the gene fusions. Exemplary prostate cancer markers include, but are not limited to: EZH2 (U.S. Pat. No. 7,229,774), AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); and, those disclosed in U.S. Pat. Nos. 5,854,206 and 6,034,218, and U.S. Publication No. 20030175736, each of which is herein incorporated by reference in its entirety. Markers for other cancers, diseases, infections, and metabolic conditions are also contemplated for inclusion in a multiplex or panel format.

The diagnostic methods of the present invention may also be modified with reference to data correlating particular gene fusions with the stage, aggressiveness or progression of the disease or the presence or risk of metastasis. Ultimately, the information provided by the methods of the present invention will assist a physician in choosing the best course of treatment for a particular patient.

A. Sample

Any patient sample suspected of containing loss or decrease in expression of miR-101 may be tested according to the methods of the present invention. By way of non-limiting examples, the sample may be tissue (e.g., a prostate or other tissue biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

The patient sample may require preliminary processing designed to isolate or enrich the sample for miRNA or genomic DNA encoding miRNA. A variety of techniques known to those of ordinary skill in the art may be used for this purpose, including but not limited: centrifugation; immunocapture; cell lysis; and, nucleic acid target capture (See, e.g., EP Pat. No. 1 409 727, herein incorporated by reference in its entirety).

B. DNA and RNA Detection

Loss or decrease in expression of miR-101 may be detected as loss of genomic DNA encoding mir-101 or decrease or absence of expression of miR-101 RNA. A variety of nucleic acid techniques known to those of ordinary skill in the art may be utilized to detect miR-101 or other nucleic acids, including but not limited to: nucleic acid sequencing; nucleic acid hybridization; and, nucleic acid amplification.

In some embodiments, a decrease in expression is determined by comparing expression level to a control (e.g., individual or average or individuals not diagnosed with cancer) or threshold value (e.g., population average, values from the same subject determined at a different time, etc.).

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

a. FISH

In some embodiments, nucleic acids are detected using fluorescence in situ hybridization (FISH). In some embodiments, FISH assays utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

These probes, or those described above, are labeled with appropriate fluorescent or other markers and then used in hybridizations. Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art to establish procedural steps convenient for a particular laboratory.

b. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Genomic DNA and miR-101 RNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Q13 replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified nucleic acids can be detected by any conventional means. For example, nucleic acids can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

C. Protein Detection

In some embodiments, cancer marker proteins (e.g., EZH2 or other cancer markers) are detected. In some embodiments, proteins are detected using immunoassays. Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

D. Data Analysis

In some embodiments, a computer-based analysis program is used to translate the raw data generated by the detection assay (e.g., the presence, absence, or amount of miR-101 or other markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient.

The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

F. Compositions & Kits

Compositions for use in the diagnostic methods of the present invention include, but are not limited to, probes, amplification oligonucleotides, and antibodies.

Any of these compositions, alone or in combination with other compositions of the present invention, may be provided in the form of a kit. For example, the single labeled probe and pair of amplification oligonucleotides may be provided in a kit for the amplification and detection of miR-101. Kits may further comprise appropriate controls and/or detection reagents. The probe and antibody compositions of the present invention may also be provided in the form of an array.

### II. Therapeutic Applications

In some embodiments, the present invention provides methods of restoring miR-101 function (e.g., to treat or prevent cancer), for example, by providing miR-101 through genetic therapy.

Delivery of nucleic acid construct to cells in vitro or in vivo may be conducted using any suitable method. A suitable method is one that introduces the nucleic acid construct into the cell such that the desired event occurs (e.g., expression of miR-101).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Appl. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subject in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

### III. Antibodies

The present invention provides isolated antibodies. In some embodiments, the present invention provides monoclonal antibodies that specifically bind to an isolated polypeptide comprised of at least five amino acid residues of EZH2 or other cancer markers. These antibodies find use in the diagnostic, therapeutic and drug screening methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### IV. Drug Screening Applications

In some embodiments, the present invention provides drug screening assays (e.g., to screen for anticancer drugs). The screening methods of the present invention utilize cancer markers identified using the methods of the present invention (e.g., including but not limited to, miR-101, alone or in combination with EZH2). For example, in some embodiments, the present invention provides methods of screening for compounds that alter (e.g., increase) the expression of miR-101. The compounds or agents may enhance transcription of miR-101, by interacting, for example, with the promoter region. In other embodiments, genetic therapies (see above) that restore miR-101 function, expression level or presence are utilized.

The compounds or agents may interfere with pathways that are upstream or downstream of the biological activity of miR-101. In some embodiments, candidate compounds are antisense or interfering RNA agents (e.g., oligonucleotides) directed against cancer markers. In other embodiments, candidate compounds are antibodies or small molecules that specifically bind to an upstream regulator of miR-101 and thus enhance the expression or activity of miR-101.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

### EXPERIMENTAL

The following examples are provided in order to demonstrate and further illustrate certain preferred embodiments and aspects of the present invention and are not to be construed as limiting the scope thereof.

### Example 1

### A. Materials and Methods

microRNA Prediction Tools

To identify miRNAs targeting EZH2, the output results of multiple prediction programs including TargetScan (Lewis et al., Cell 115, 787 (Dec. 26, 2003)), PicTar (Krek et al., Nat Genet. 37, 495 (May, 2005)), miRanda (John et al., PLoS Biol 2, e363 (November, 2004)), and miRInspector (Rusinov et al., Nucleic Acids Res 33, W696 (Jul. 1, 2005)) were utilized. Each program was selected to leverage the various strengths for predicting miRNA targets in the areas of sequence alignment, thermodynamics, and comparative genomics. TargetScan requires a perfect seed, a sub-region of alignment between the miRNA and mRNA, while incorporating traditional RNA folding calculations and conservation of the binding site across vertebrates. PicTar, while preferring a perfect seed match, tolerates imperfect seed matches when they simultaneously adhere to heuristically defined thermodynamic requirements. miRanda employs a dynamic programming algorithm to establish miRNA:mRNA sequence alignment in addition to thermodynamics and conservation across multiple species. Lastly, microInspector identifies possible binding sites within an mRNA sequence relying heavily on free energy values at a binding site. A Perl script that imported the various output formats from each of the target prediction programs was developed and the results were interrogated to detect common overlaps. For instance where all programs report an miRNA:mRNA interaction, the candidate miRNAs are sorted based on the predefined rankings from each respective program. Additionally, the number of predicted binding sites for miR-101 in the EZH2 3′UTR was exported.

### Cell Lines

Breast cancer cell line SKBr3 were grown in RPMI 1640 (Invitrogen, Carlsbad, Calif.) with 10% FBS (Invitrogen) in 5% CO₂ cell culture incubator, and prostate cancer cell line DU145 were grown in MEM with 10% FBS in 5% CO2 cell culture incubator. Immortalized breast cell lines HME and H16N2 were grown in F-12 Nutrient Mixture with 5 μg/ml Insulin (Sigma), 1 μg/ml Hydrocortisone (Sigma), 10 ng/ml EGF Invitrogen), 5 mM Ethanolamine (Sigma), 5 μg/ml Transferrin (Sigma), 10 nM Triiodo Thyronine (Sigma), 50 nM Sodium Selenite (Sigma), 10 mM HEPES (Invitrogen) and 50 unit/ml Penstrep (Invitrogen), 10% CO₂. For mir-101 overexpression, pMIF-cGFP-Zeo construct expressing mir-101 was obtained from System Biosciences (Mountain View, Calif.). Lentiviruses were generated by the University of Michigan Vector Core. Prostate cancer cell line DU145 and breast cancer cell line SKBR3 were infected with lentiviruses expressing mir-101 or vector only, and stable cell lines were generated by selection with 300 μg/ml zeocin (Invitrogen, Carlsbad, Calif.). To generate stable EZH2 knockdown, shRNA lentiviral particles for EZH2 gene silencing and control vector were obtained from Sigma-Aldrich (St. Louis, Mo.). Prostate cancer cell line DU145 was infected with EZH2 shRNA lentivirus and a stable cell line was generated by selection with 1 μg/ml Puromycin (Sigma-Aldrich, St. Louis, Mo.).

### Tissues

In this study tissues from clinically localized prostate cancer patients, who underwent radical prostatectomy as a primary therapy between 2004-2006 at the University of Michigan Hospital, androgen-independent metastatic prostate cancer patients from a rapid autopsy program and patients with invasive carcinomas of the breast were utilized. The detailed clinical and pathological data were maintained on a secure relational database. Both radical prostatectomy series and the rapid autopsy program were part of the University of Michigan Prostate Cancer Specialized Program of Research Excellence Tissue Core. Breast cancer tissues were collected with IRB approval from the University of Singapore/National University Hospital, Singapore (NUS/NUH). The gastric cancer samples were collected with IRB approval from the National Cancer Center, Singapore.

microRNA Transfection, AntagomiR Transfection, and Small RNA Interference

Knockdown of EZH2 was accomplished by RNA interference using siRNA duplex (Dharmacon, Lafayette, Colo.) as previously described (Varambally et al., Nature 419, 624 (2002)). Precursors of respective microRNAs and negative controls used in this study were purchased from Ambion (Austin, Tex.). AntagomiR-101 and negative control antagomiRs were purchased from Dharmacon. Transfections were performed with oligofectamine or lipofectamine (Invitrogen) depending on the cell line used.

### miR Reporter Luciferase Assays

The 3′UTR (untranslated region) or the antisense sequence of the 3′UTR of EZH2 as well as mutant 3′UTR of EZH2 were cloned into the pMIR-REPORT™ miRNA Expression Reporter Vector (Ambion). SKBr3 cells were transfected with pre-miR-101 or controls and then co-transfected with 3′-UTR-luc or mutant 3′UTR-luc, as well as pRL-TK vector as internal control for luciferase activity. Post 48 hours of incubation, the cells were lysed and luciferase assays conducted using the dual luciferase assay system (Promega, Madison, Wis.). Each experiment was performed in triplicate.

### Quantitative Real-Time PCR Assays

Total RNA was isolated from SKBr3 and DU145 cells that were transfected either with pre-miR-101, or control precursors (Qiagen). Quantitative PCR (QPCR) was performed using SYBR Green dye on an Applied Biosystems 7300 Real Time PCR system (Applied Biosystems, Foster City, Calif.) as described (Tomlins et al., Science 310, 644 (Oct. 28, 2005)). Briefly, 1 μg of total RNA was reverse transcribed into cDNA using SuperScript III (Invitrogen, Carlsbad, Calif.) in the presence of random hexamers and oligo dT primers (Invitrogen). All reactions were performed in triplicate with SYBR Green Master Mix (Applied Biosystems) plus 25 ng of both the forward and reverse primer according to the manufacturer's recommended thermocycling conditions, and then subjected to melt curve analysis. Threshold levels for each experiment were set during the exponential phase of the QPCR reaction using Sequence Detection Software version 1.2.2 (Applied Biosystems). The DNA in each sample was quantified by interpolation of its threshold cycle (Ct) value from a standard curve of Ct values, which were created from a serially diluted cDNA mixture of all samples. The calculated quantity of the target gene for each sample was divided by the average sample quantity of the housekeeping genes, glyceraldehyde-3-phosphate dehydrogenase (GAPDH) to obtain the relative gene expression. All oligonucleotide primers were synthesized by Integrated DNA Technologies (Coralville, Iowa). The primer sequences for the transcript analyzed are provided in Table 4.

For microRNA quantitative PCR, total RNA including small RNA was isolated from prostate tissues, SKBr3 and DU145 cells that were transfected either with pre-hsa-miR-101 (precursor human miRNA-101), or control precursors. Total RNA was used at 10 ng/μl. For RT, master mix were prepared using 0.15 μl 100 mM dNTPs, 1.00 μl MultiScrible Reverse Transcriptase (50 U/μl), 1.50 μl 10× Reverse Transcription Buffer, 0.188 μl RNase Inhibitor (20 U/μl) and 4.192 μl Nuclease-free water. Each 15 μl RT reaction mix contained 7 μl of master mix, 5 μl of RNA samples (10 ng/μl) and 3 μl 5× specific RT primer. Thermal cycler was programmed as follows: 16 degrees for 30 minutes, 42 degrees for 30 minutes and 85 degrees for 5 minutes. Each PCR reaction mix contained 10 μl of Taqman 2× Universal PCR Master Mix (No AmpErase UNG), 6.67 μl Nuclease-free water, 1 ul 20× specific PCR primer and 1.33 ul RT product. Thermal cycler was programmed as follows: 95 degrees for 10 minutes, 40 cycles of 95 degrees for 15 seconds and 60 degrees for 60 seconds. Using the comparative CT method, endogenous control (RNU6B) was used to normalize the expression levels of target micro-RNA by correcting differences in the amount of RNA loaded into qPCR reactions.

### Genomic PCR Assays

Genomic DNA from benign (n=15), localized prostate cancer (n=16) and metastatic (n=33) prostate cancer tissues were isolated. DNA from benign (n=7), tumor (n=29) and metastatic (n=1, three different sites) from breast cancer cases were also isolated. For genomic analyses of the miR-101 loci, the 2̂ΔΔCt method was adapted using SyBr green based quantitative PCR (qPCR) (Ferreira et al., Malar J 5, 1 (2006); Malakho et al., J Clin Lab Anal 22, 123 (2008). Briefly, 100 ng 25 ng of gDNA was used as template to amplify the miR-101-1, miR-101-2 and miR-217 encompassing loci. Since miR-217 levels did not show significant correlation with EZH2 transcript levels, miR-217 was used as the reference for relative quantification. The assay was validated using the methods described (Ferreira et al., supra; Malakho et al., supra). For unification of data and to avoid inter-assay differences Ct values for the reference gene (miR-217) were always estimated simultaneously with miR-101-1 or miR-101-2. A representative benign tissue sample was used in every assay as a calibrator sample to which every sample was compared, to obtain a relative quantitation (RQ) value. To calibrate the extent of loss in the miR-101 loci the relative levels of 9 different genomic regions on X-chromosome were determined (three regions in phosphoglycerate kinase 1 (PGK1) gene, and six X-chromosome specific miRNAs-miR-424, miR-503, miR-766, miR-448, miR-222 and miR-221) in the genomic DNA from a normal male sample (1×) as compared to a normal female sample (2×) genomic DNA (Promega) that are located only on the X chromosome. RQ values for these regions in male genomic DNA were assessed using a non-X chromosome gene Tata Binding Protein (TBP) gene as the reference gene (Malakho et al., supra). An R^{Q} value of 0.7 and below was considered as loss of at least one copy of the genomic loci (Table 5), similar to earlier reports (Ferreira et al., supra; Malakho et al., supra). Accordingly, samples showing values lower than 0.7 were considered to have a hemizygous loss and those below 0.3 were considered to exhibit a homozygous loss. For the loss of heterozygosity (LOH) analysis, 9 cancer samples showing miR-101-1 deletion were identified and normal (non prostatic) tissues from the same cases were obtained. Genomic qPCR analysis was carried out in these as described above and RQ values obtained were compared to those obtained from the matched cancer cases. Primer sequences used for genomic PCR assays are given in Table 6.

### Immunoblot Analyses

The breast cancer cell lines SKBr3 and prostate cancer cell DU145 were transfected with pre-miR-101 or controls. The breast cell lines H16N2 and. HME were transfected with antagomiR-101 or negative controls. Post 72 hours transfection, cells were homogenized in NP40 lysis buffer (50 mM Tris-HCl, 1% NP40, pH 7.4, Sigma, St. Louis, Mo.), and complete proteinase inhibitor mixture (Roche, Indianapolis, Ind.). Ten micrograms of each protein extract were boiled in sample buffer, separated by SDS-PAGE, and transferred onto Polyvinylidene Difluoride membrane (GE Healthcare, Piscataway, N.J.). The membrane was incubated for one hour in blocking buffer [Tris-buffered saline, 0.1% Tween (TBS-T), 5% nonfat dry milk] and incubated overnight at 4° C. with the following: anti-EZH2 mouse monoclonal (1:1000 in blocking buffer, BD Biosciences Cat # 612667, San Jose, Calif.), anti-EED rabbit polyclonal (1:1000, Santa Cruz Biotech, Cat #: sc-28701, Santa Cruz, Calif.), anti-SUZI2 mouse monoclonal (1:1000, Upstate, Cat #: 04-046, Charlottesville, Va.), and anti-N-myc rabbit polyclonal antibodies (1:1000, Cell Signaling Tech, Cat #: 9405, Danvers, Mass.), anti-ARID1A mouse monoclonal antibody (1:1000, Abcam, Cat #: ab50878, Cambridge, Mass.), anti-FBN2 rabbit polyclonal antibody (1:1000, Abcam, Cat #: ab21619), anti-c-Fos mouse monoclonal antibody (1:1000, BD Biosciences, Cat #: 554156), antitrimethyl-H3K27 rabbit polyclonal (1:2000, Upstate, Cat #: 07-449), anti-monomethyl-Histone H3 (Lys27) (1:1000 upstate Cat #: 07-448), anti-acetyl-Histone H3 (K27) (upstate Cat #: 07-360) and antitotal Histone H3 rabbit polyclonal (1:5000, Cell Signaling, Cat #: 9715) and anti-GAPDH mouse monoclonal antibody (1:10000, Abcam, Cat #: ab9482). Following a wash with TBS-T, the blot was incubated with horseradish peroxidase-conjugated secondary antibody and the signals visualized by enhanced chemiluminescence system as described by the manufacturer (GE Healthcare).

### Cell Proliferation Assay

Cells were plated in 24-well plates at desired cell concentration and transfected with precursor microRNA or controls. Cell counts were estimated by trypsinizing cells and analysis by Coulter counter (Beckman Coulter, Fullerton, Calif.) at the indicated time points in triplicate.

### Cell Migration Assay Using Wound Healing Assay

DU145 lenti-vector and miR-101 overexpressing, and sh-vector and EZH2 knockdown stable cells were grown to confluence. An artificial wound was created using a 1 ml pipette tip on confluent cell monolayer. To visualize migrated cells and wound healing, cell images were taken at 0, 24, 48 and 72 hrs.

### Basement Membrane Matrix Invasion Assays

For invasion assays, the breast cell lines H16N2 and HME were transfected with antagomiR-101 or negative controls. Invasive breast cancer cell SKBr3 and prostate cancer cell DU145 were transfected with pre-miR-101 or controls. Forty-eight hours post-transfection, cells were seeded onto the basement membrane matrix (EC matrix, Chemicon, Temecula, Calif.) present in the insert of a 24 well culture plate. Fetal bovine serum was added to the lower chamber as a chemoattractant. After 48 hours, the non-invading cells and EC matrix were gently removed with a cotton swab. Invasive cells located on the lower side of the chamber were stained with crystal violet, air dried and photographed. For colorimetric assays, the inserts were treated with 150 μl of 10% acetic acid and the absorbance measured at 560 nm using a spectrophotometer (GE Healthcare).

### Soft Agar Colony Formation Assays

A 50 μL base layer of agar (0.6% Agar in DMEM with 10% FBS) was allowed to solidify in a 96-well flat-bottom plate prior to the addition of a 75 pit wild type DU145, DU145 miR-101 clones or vector transfected DU145 cell suspension containing 4,000 cells in 0.4% Agar in DMEM with 10% FBS. The cell containing layer was then solidified at 4° C. for 15 minutes prior to the addition of 100 μL of MEM with 5% FBS. Colonies were allowed to grow for 21 days before imaging under a light microscope.

### Prostate Tumor Xenograft Model

All procedures involving mice were approved by the University Committee on Use and Care of Animals (UCUCA) at the University of Michigan and conform to their relevant regulatory standards. Five-week old male nude athymic BALB/c nu/nu mice (Charles River Laboratory, Wilmington, Mass.) were used for examining the tumorigenicity. To evaluate the role of miR-101 overexpression in tumor formation, the DU145 stable cells overexpressing miR-101 or vector control cells were propagated and 5×10⁶ cells were inoculated subcutaneously into the dorsal flank of ten mice (n=5 per group). Tumor size was measured every week, and tumor volumes were estimated using the formula (π/6) (L×W2), where L=length of tumor and W=width.

### Chromatin Immunoprecipitation (ChIP) Assays.

The effect of miR-101 over-expression on trimethyl H3 (Lys-27) status of EZH2 targets was determined by Chromatin Immunoprecipitation (ChIP) assay. The ChIP assay was carried out with antibodies against trimethyl H3 (Lys-27) (Mouse monoclonal from Abcam, Cat: Ab6002-100). The assay was performed using the EZ-Magna ChIP kit (Millipore) according to the manufacturer's protocol. Briefly, 2×10⁶ cells were used for each immunoprecipitation. The cells were cross-linked for 10 minutes by addition of formaldehyde to a final concentration of 1%. The cross-linking was stopped by 1/20V of 2.5M glycine. This was followed by cell lysis and sonication, resulting in an average fragment size of 500 bp. Antibody incubations were carried out over-night at 4° C. Reversal of cross-linking was carried out at 65° C. for 3 hours, followed by DNA isolation. The purified DNA was analyzed by quantitative PCR to determine fold enrichment relative to input DNA. The primer sequences for the promoters analyzed are provided in Table 7.

### Gene Expression Profiling

Expression profiling was performed using the Agilent Whole Human Genome Oligo Microarray (Santa Clara, Calif.) according to the manufacturer's protocol. SKBr3 cells were transfected with pre-miR-101 or negative control for precursor microRNA. Over- and under-expressed signatures were generated by filtering to include only features with significant differential expression (Log ratio, P<0.01) in all hybridizations and two-fold average over- or under-expression (Log ratio) after correction for the dye flip. To ensure that we were comparing robust gene expression alterations, biological replicates were analyzed and only the probes showing expression changes in both replicates were used.

### Array Comparative Genomic Hybridization

Comparative genomic hybridization analysis for prostate, breast and gastric cancers were carried out using oligonucleotide based comparative genomic hybridization array (Hg17 genome build) (Agilent Technologies, USA) according the manufacture's instructions. Competitive hybridization of differentially labeled tumor and reference DNA to oligonucleotide printed in an array format and analysis of fluorescent intensity for each probe will detect the copy number changes in the tumor sample relative to normal reference genome. Copy number changes were analyzed for miR-101-1 (1p31.3) and miR-101-2 (9p24.1) regions with a change in copy number level of at least one copy (log ratio±0.5) for losses involving more than one probe representing each genomic interval as detected by the aberration detection method (ADM-2) in CGH analytics software 3.5 (Agilent Technologies) algorithm.

Analysis of Publicly Available Array CGH/SNP Datasets for miR-101 Copy Number Analysis

To examine the mir-101 loss status in multiple cancers, the public array CGH/SNP datasets from Gene Expression Omnibus and Cancer Bioinformatics Grid were utilized. Acute lymphoblastic leukemia (Mullighan et al., Nature 446, 758 (Apr. 12, 2007)), glioblastoma (Data from TCGA) and lung cancer (Zhao et al., Cancer Res 65, 5561 (Jul. 1, 2005)) studies were analysed. The sample information was manually curated and classified into cancer (primary plus metastasis), metastasis and normal samples.

For the Affymetrix SNP arrays, model-based expression was performed to summarize signal intensities for each probe set, using the perfect-match/mismatch (PM/MM) model. For copy number inference, raw copy number was calculated by comparing the signal intensity of each SNP probe set for each tumor sample against a diploid reference set of samples. All of the resulting DNA copy number ratios were transformed by log 2. In the two-channel array CGH datasets, the differential ratio between the processed testing channel signal and processed reference channel signal were calculated and transformed by log 2, which reflects the DNA copy number difference between the testing channel and reference channel.

In the normalization step, the log ratios were transformed into a normal distribution with a mean of 0 under the null model assumption. The data were segmented by circular binary segmentation (CBS) algorithm developed by Olshen et al, (Olshen et al., Biostatistics 5, 557 (October, 2004)) a method for identifying all genomic change points where the mean log ratio score changes between intervals. The threshold for deletion was modified from the report by Mullighan et al, (Mullighan et al., supra). Cutoffs of 0.9 and 0.3 were used to identify hemizygous and homozygous deletions, respectively. The probe closest to the selected gene was used to represent the DNA copy number status of this gene, with a maximal distance of 10 kb.

### Statistical Analyses

All gene expression and relative quantification data were analyzed on the log (base 2) scale. Comparisons between gene expression values across sample classes were made using two-sample Student's t-test. The significance of associations between EZH2 and miR-101 expression values was judged via a test statistic based on Pearson's product moment correlation coefficient. Associations between binary variables (loss at the two miR-101 loci, and overlap of gene sets) were explored using Fisher's exact test. The relationship between EZH2 overexpression and miR-101 loss was evaluated using a test statistic calculated as the minimum observed value of EZH2 expression in the set of samples exhibiting miR-101 loss at either locus. The null permutation distribution of this statistic was derived by randomly permuting miR-101 loss status within the set of samples; N=10000 permutations were used.

The significance of the separation between miR-101 and vector trajectories in the mouse xenograft model was evaluated via a linear mixed model that incorporated a random intercept for each mouse and used square-root transformed tumor volume measurements as dependent variable. Wald tests were used to assess the statistical significance of observed differences between growth rates in the two groups of mice.

All statistical tests were two-sided and constructed at the a=0.05 significance level except for the above described permutation test, which was one-sided and conducted at the α=0.025 significance level. Statistical analyses were performed using R, version 2.7.0.

### B. Results

Polycomb Group Proteins, including EZH2, play a master regulatory role in controlling important cellular process such as maintaining stem cell pluripotency (Boyer et al., Nature 441, 349 (2006); Lee et al., Cell 125, 301 (2006); Sher et al., Stem Cells (2008)), cell proliferation (Varambally et al., Nature 419, 624 (2002); Bracken et al., EMBO J. 22, 5323 (2003)), early embryogenesis (Erhardt et al., Development 130, 4235 (2003)), and X chromosome inactivation (Plath et al., Science 300, 131 (2003)). EZH2 functions in a multi-protein complex called Polycomb Repressive Complex 2 (PRC2) which includes SUZ12 (Suppressor of Zeste 12) and EED (Embryonic Ectoderm Development) (Cao et al., Science 298, 1039 (2002); Kuzmichev et al., Genes Dev. 16, 2893 (2002)). The primary activity of the EZH2 protein complex is to tri-methylate histone H3 lysine 27 (H3K27) at target gene promoters, leading to epigenetic silencing (J. Yu et al., Cancer Cell 12, 419 (2007); Cao et al., Oncogene (2008). Mounting evidence indicates that EZH2 has properties consistent with those of an oncogene, as overexpression promotes cell proliferation, colony formation, and increased invasion of benign cells in vitro (Varambally et al., supra, Bracken et al., supra, Kleer et al., Proc. Natl. Acad. Sci. U.S.A. 100, 11606 (2003)) and induces xenograft tumor growth in vivo (Croonquist, B. Van Ness, Oncogene 24, 6269 (2005)). Likewise, knock-down of EZH2 in cancer cells results in growth arrest (Varambally et al., supra Croonquist et al., supra) as well as diminished tumor growth (Yu et al., supra) and metastasis in vivo (Takeshita et al., Proc. Natl. Acad. Sci. U.S.A. 102, 12177 (2005)). EZH2 was initially found to be elevated in a subset of aggressive, clinically localized prostate cancers and almost all metastatic prostate cancers (Varambally et al., supra). Subsequently EZH2 has also been found to be aberrantly overexpressed in breast cancer (Kleer et al., supra), melanoma (Bachmann et al., J. Clin. Oncol. 24, 268 (2006)), bladder cancer (Weikert et al., Int. J. Mol. Med. 16, 349 (2005)), gastric cancer (Matsukawa et al., Cancer Sci. 97, 484 (2006)) and other cancers (Bracken et al., supra). Thus, while EZH2 is broadly overexpressed in aggressive solid tumors and has properties of an oncogene, the genetic mechanism of EZH2 elevation in cancer is unclear.

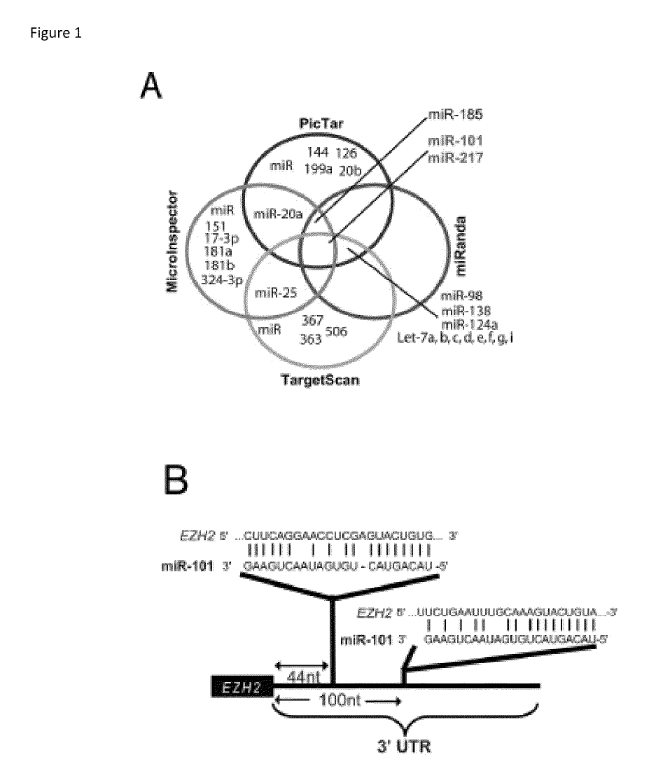

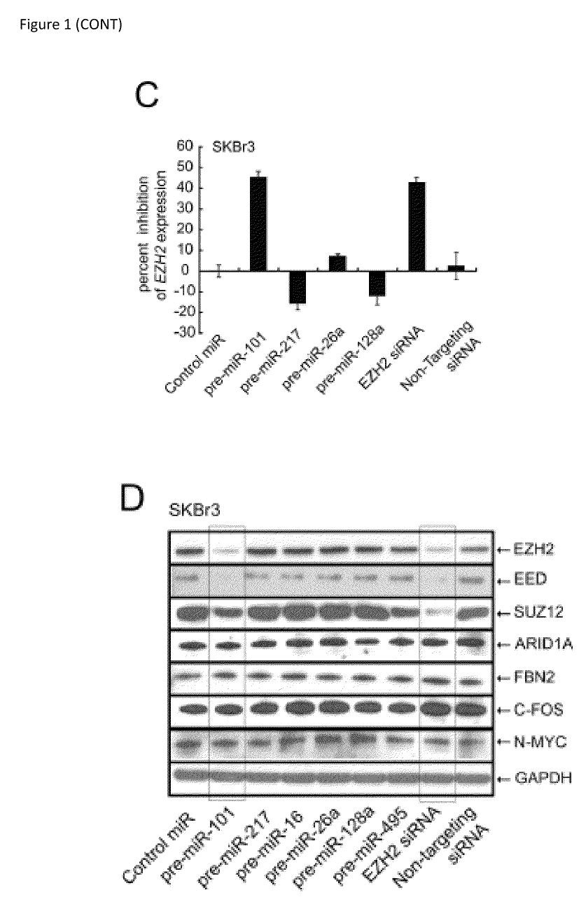

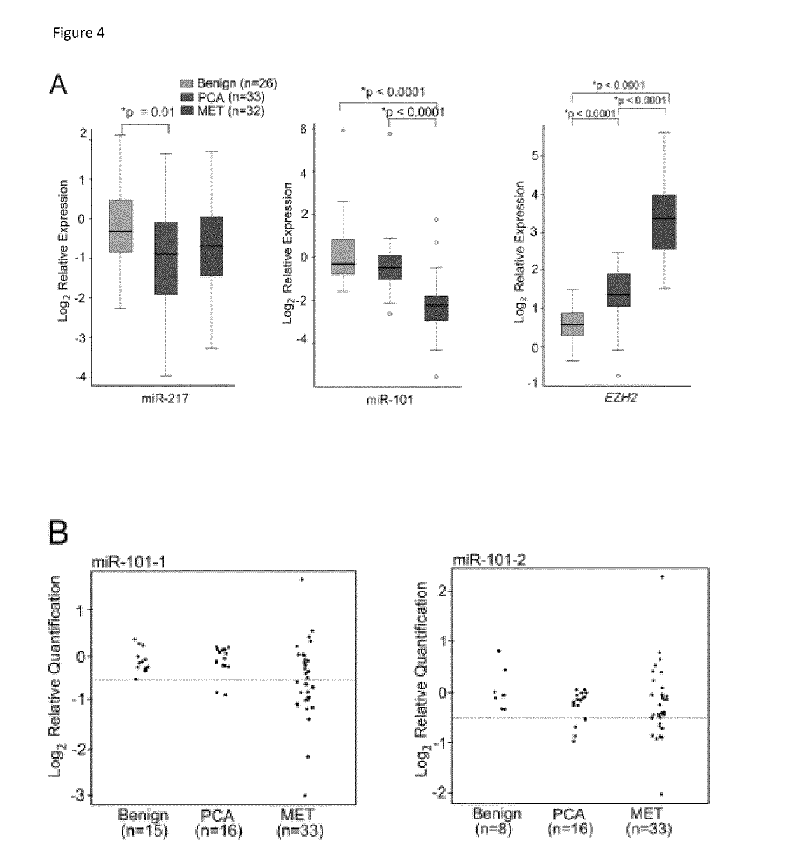

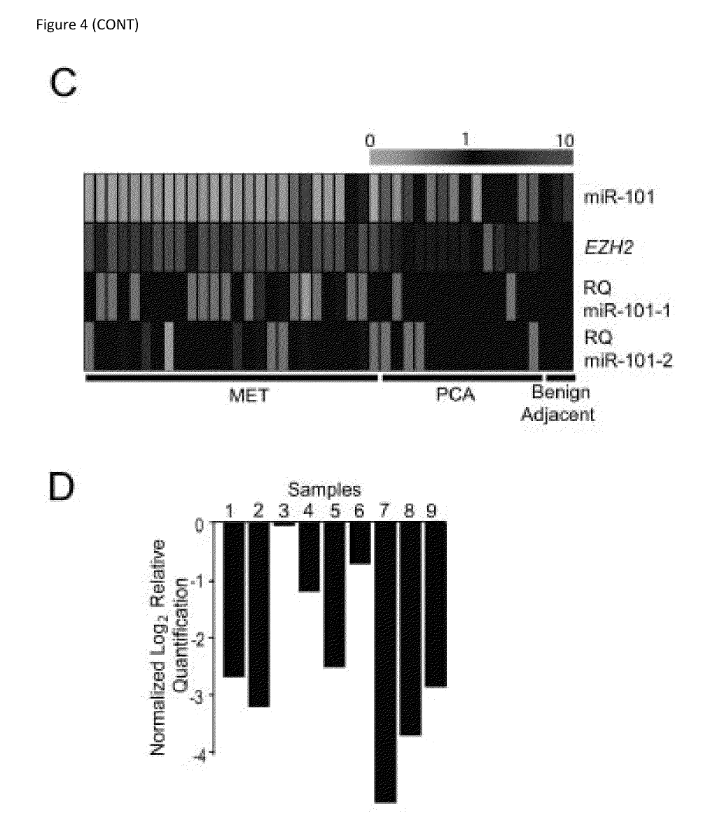

As microRNAs (miRNAs) have gained considerable attention as regulators of gene expression (Lewis et al., Cell 120, 15 (2005)), and play important roles in cellular differentiation and embryonic stem cell development (Marson et al., Cell 134, 521 (Aug. 8, 2008)), it was contemplated that they play a role in modulating EZH2 expression. To test whether miRNAs play a role in controlling EZH2 expression, computer modeling was used to identify those that might contribute to EZH2 regulation. Because intersecting the results of multiple prediction algorithms can increase specificity at the cost of lower sensitivity (Sethupathy et al., Nat. Methods 3, 881 (2006)), the results of the prediction programs PicTar (Krek et al., Nat. Genet. 37, 495 (2005)), TargetScan (Lewis et al., Cell 115, 787 (2003)), miRanda (John et al., PLoS Biol. 2, e363 (2004)), and miRInspector (Rusinov et al., Nucleic Acids Res. 33, W696 (2005)) were investigated. Overall, only 29 miRNAs were found by any program to target EZH2, while only miR-101 and miR-217 were found by all four programs to be predicted to regulate EZH2 (FIG. 1A and Table 1). Furthermore, PicTar, miRanda, and TargetScan predicted two miR-101 binding sites within the EZH2 3′UTR (FIG. 1B) whereas PicTar and TargetScan predicted two miR-217 binding sites within the EZH2 3′UTR. Of the 34 miRNAs predicted to regulate EZH2 by at least one program (Table 1), only miR-101 had a strong negative association with prostate cancer progression from benign to localized disease to metastasis (FIG. 4A).

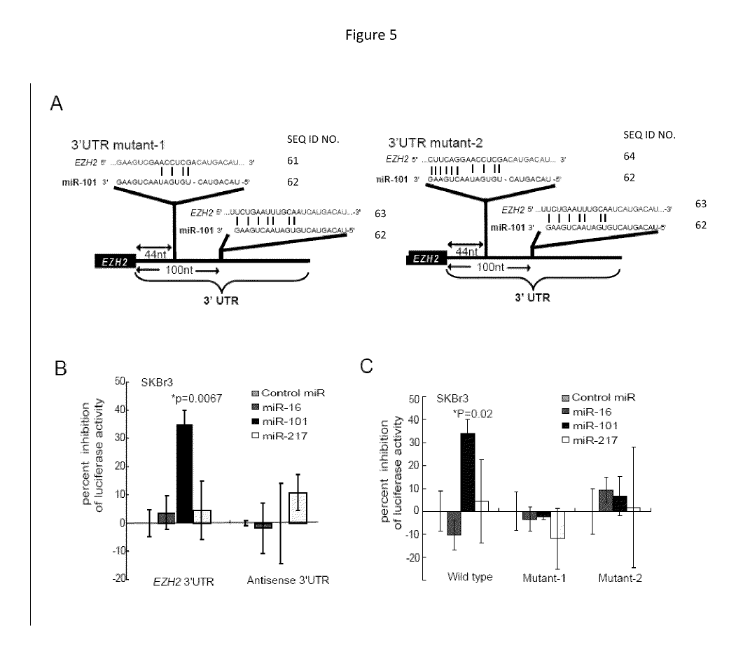

To examine whether miR-101 regulates the 3′UTR of EZH2, luciferase reporters encoding the normal, antisense, and mutated versions of the EZH2 3′UTR were generated. Overexpression of miR-101, but not miR-217 or control miRNA decreased the activity of the luciferase reporter encoding the 3′UTR of EZH2 (FIG. 5). Similarly, the antisense and mutant EZH2 3′UTR activities were not inhibited by miR-101. To explore whether the 3′UTR binding by miR-101 results in down-regulation of the EZH2 transcript, SKBr3 breast cancer cells (which express high level of endogenous EZH2) were transfected with precursors of miR-101, miR-217, control miRNA as well as several other unrelated miRNAs. qRT-PCR demonstrated a reduction in EZH2 transcript levels by miR-101 (FIG. 1C), but not miR-217 or other control miR5.

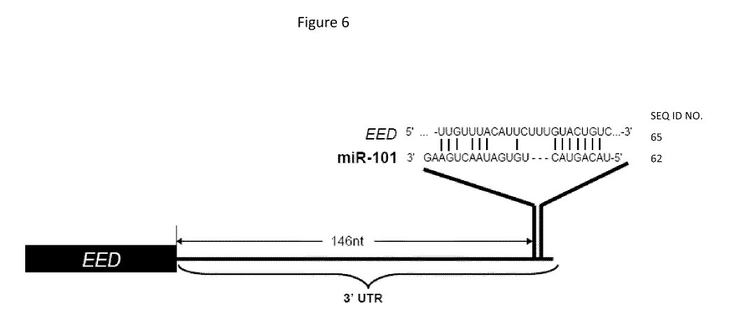

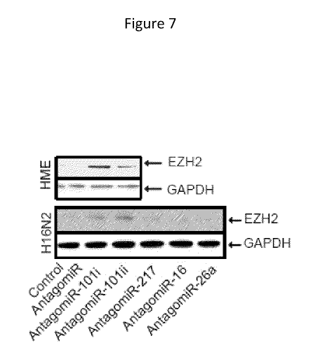

To determine whether miR-101 represses EZH2 protein expression, immunoblot analysis was performed using an EZH2 specific antibody, as well as antibodies to other PRC2 members, including EED and SUZ12 (FIG. 1D). In addition to miR-101, other miRNAs predicted to regulate EZH2, including miR-217 and miR-26a, were included. Control miR-495 was predicted by TargetScan to target PRC1 component BMI-1. Only miR-101 and EZH2 siRNA attenuated EZH2 protein expression. miR-101 overexpression also leads to repression of EZH2's tight binding partners in the PRC2 complex, EED, and to a lesser extent SUZ12. These proteins are thought to form a co-regulated functional complex and altering the expression of one affects the expression of the others (Bracken et al., EMBO J. 22, 5323 (2003); Pasini et al., EMBO J. 23, 4061 (2004); Fiskus et al., Mol. Cancer. Ther. 5, 3096 (2006)). In this particular case, upon further inspection of the 3′UTRs of the PRC2 components, miR-101 binding sites were found in EED (FIG. 6) but not in SUZ12. Since miRNAs are known to regulate multiple target genes, and in some cases hundreds of genes (Lewis et al., Cell 120, 15 (2005)), the prediction algorithm TargetScan was used to nominate targets of miR-101. In addition to EZH2 and EED, 4 miR-101 predicted targets (Table 2) that have been implicated in cancer pathways including n-Myc, c-Fos, ARID1A, and FBN2 were screened. None of these putative miR-101 targets were affected by overexpression of miR-101 (FIG. 1D). To support the findings from our miR-101 overexpression experiments, antagomiR technology (Krutzfeldt et al., Nature 438, 685 (2005)) was used to specifically inhibit miR-101 expression in benign immortalized breast epithelial cells (FIG. 7). Two independent antagomiRs to miR101 (i and ii) induced expression of EZH2 protein in benign breast epithelial cells.

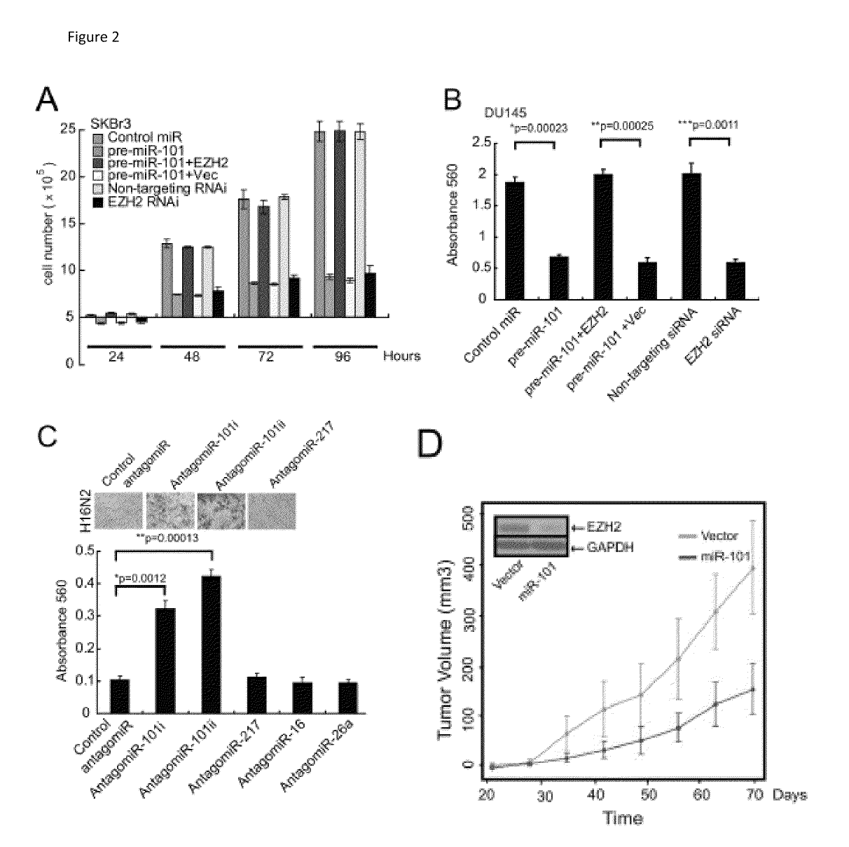

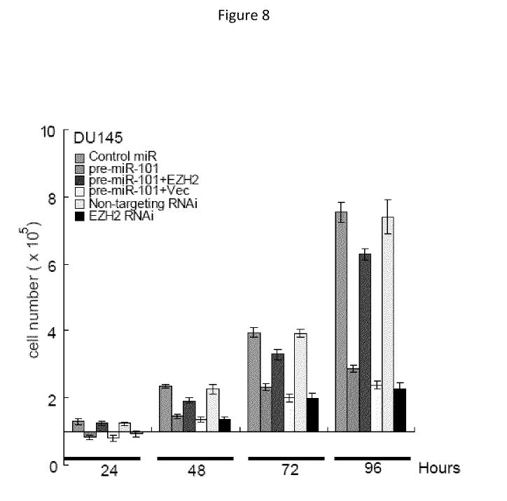

To determine whether miR-101 affects EZH2 and PRC2 function, cellular proliferation, a property known to be regulated by EZH2, was evaluated (Varambally et al, supra; Bracken et al., supra). miR-101 overexpression in SKBr3 and DU145 cells markedly attenuated cell proliferation (FIG. 2A, FIG. 8). Overexpression of EZH2 (without an endogenous 3′UTR) rescued the inhibition of cell growth by miR-101, indicating target specificity.

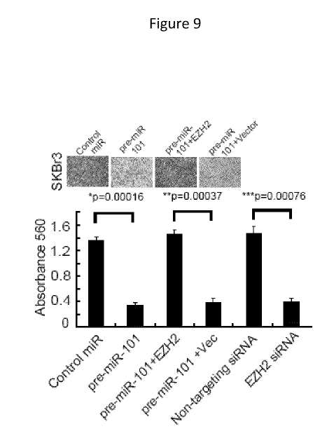

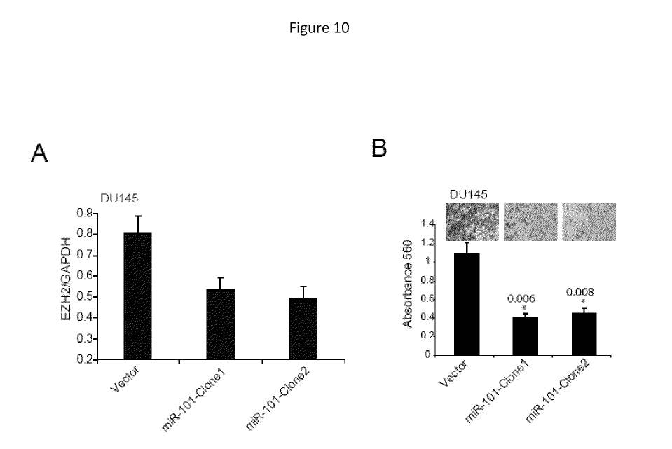

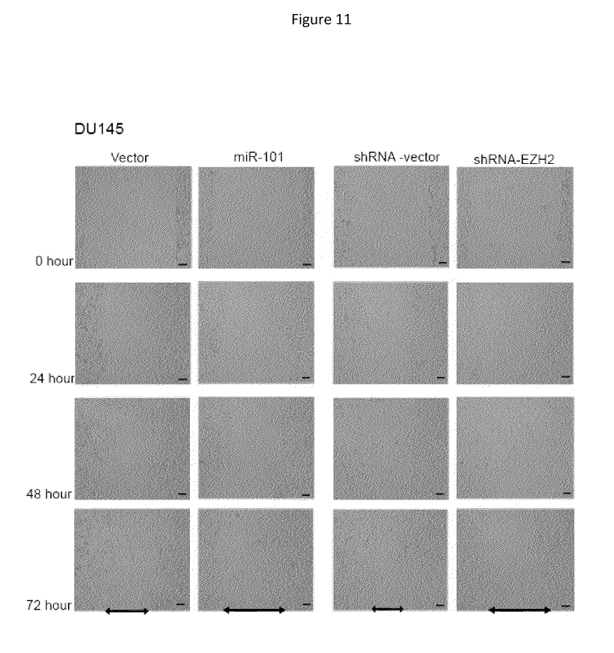

It was shown previously that upon overexpression, EZH2 can induce cell invasion in matrigel-coated basement membrane invasion assays (Kleer et al., Proc. Natl. Acad. Sci. U.S.A. 100, 11606 (2003)). Here it was show that miR-101 overexpression markedly inhibits the in vitro invasive potential of DU145 prostate cancer cells (FIG. 2B) and SKBr3 breast cancer cells (FIG. 9). Similarly, stable expression of miR-101 in DU145 cells showed a reduction in EZH2 expression and reduced invasion (FIG. 10A, B). Overexpression of EZH2 rescued the inhibition mediated by miR-101. Another in vitro readout for tumorigenic potential, increased cell migration, was also inhibited by miR-101 (FIG. 11). As overexpression of miR-101 attenuates cancer invasion, inhibition of miR-101 should enhance this neoplastic phenotype. Two independent antagomiRs targeting miR-101 (i and ii) induced an invasive phenotype when transfected into benign immortalized breast epithelial cell lines H16N2 or HME (FIG. 2C, FIG. 12).

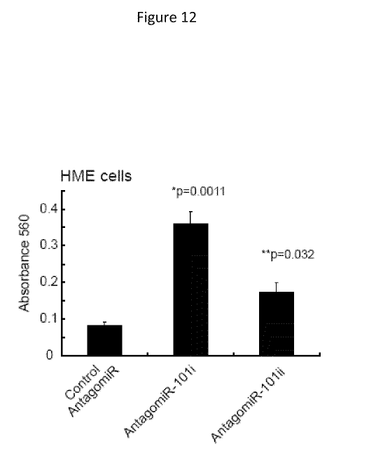

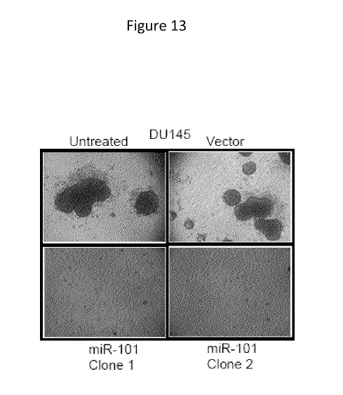

To assess whether miR-101 inhibits anchorage independent growth, a soft agar assay was used. DU145 prostate cancer cells stably overexpressing miR-101 exhibited markedly reduced colony formation relative to the parental cells or vector controls (FIG. 13). Furthermore, in vivo, DU145 cells expressing miR-101 grew significantly slower than the vector control xenografts (P=0.0001, FIG. 2D) demonstrating that miR-101 has properties consistent with that of a tumor suppressor in these particular assays.

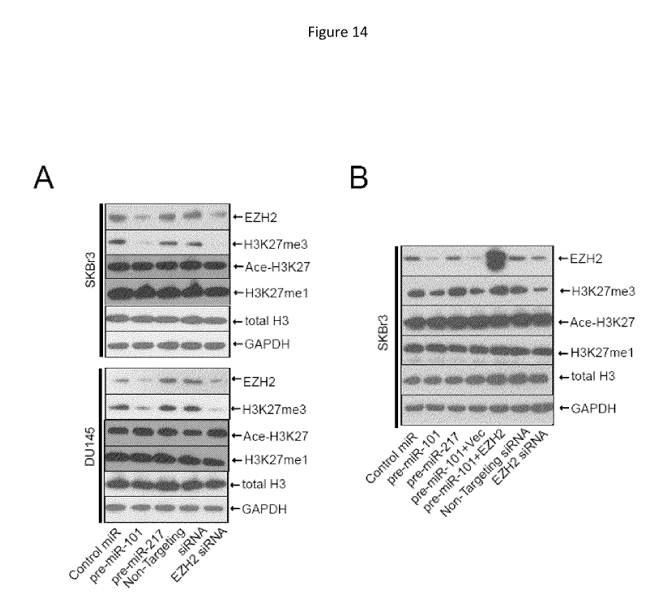

As EZH2 and PRC2 regulate gene expression by trimethylating H3K27, it was contemplated that miR-101 overexpression would result in decreased overall H3K27 trimethylation in cancer cells. SKBr3 breast cancer and DU145 prostate cancer cells transfected with miR-101 or EZH2 siRNA for 7 days displayed a global decrease in tri-methyl H3K27 levels (FIG. 14A). The effect of miR-101 on H3K27 methylation was negated by overexpression of EZH2 (FIG. 14B).

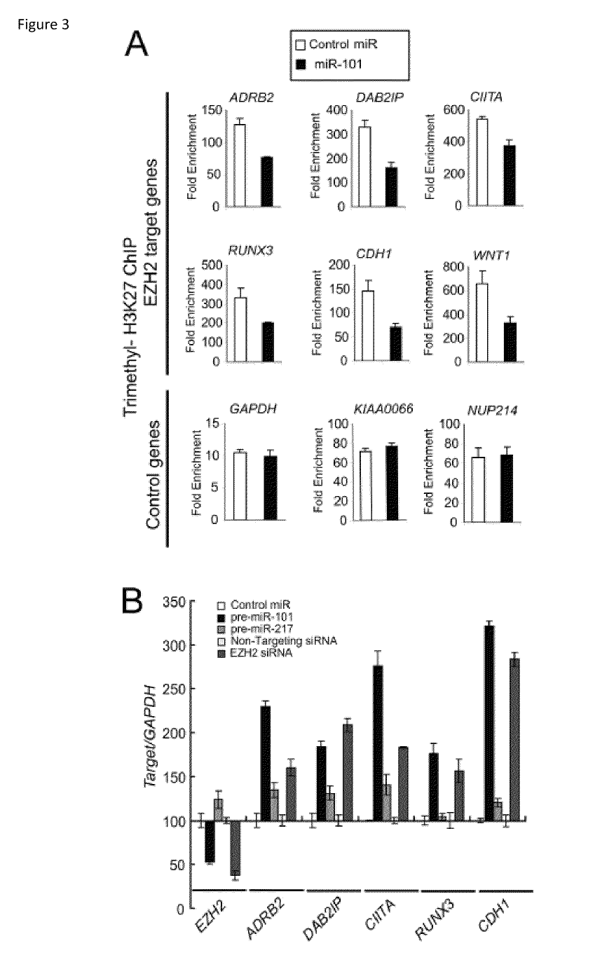

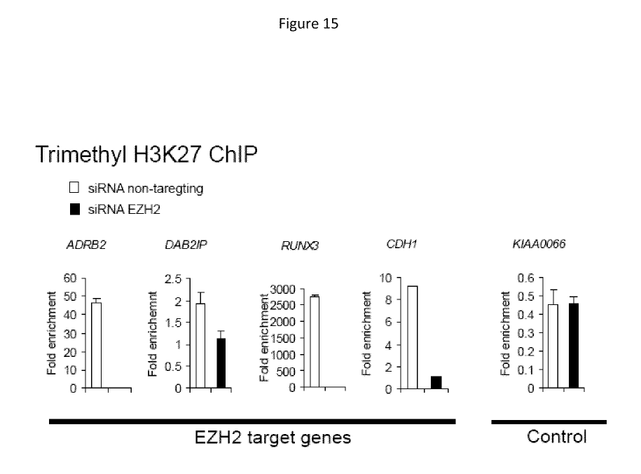

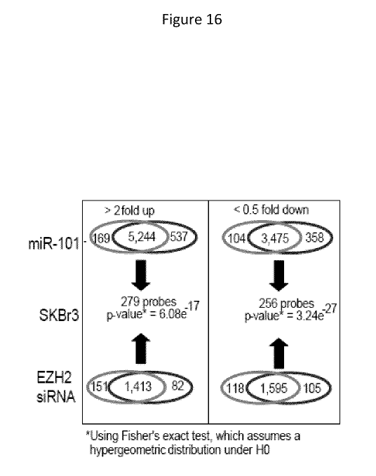

To test the level of promoter occupancy of the H3K27 histone mark, chromatin immunoprecipitation (ChIP) assays were performed in cancer cells overexpressing miR-101. Significant reduction in the tri-methyl H3K27 histone mark was found at the promoter of known PRC2 target genes such as ADRB2, DAB21P, CIITA and WNT1 in miR-101 overexpressing SKBr3 cells and EZH2 siRNA treated cells (FIG. 3A, FIG. 15). To determine whether the reduced promoter occupancy by H3K27 results in concomitant reduction of gene expression, qRT-PCR was performed on the PRC2 targets tested by ChIP assay. There was a significant increase in target gene expression in both miR-101 and EZH2 siRNA treated cells (FIG. 3B). To further explore miR-101 regulation of EZH2 and its possible similarity with EZH2 specific RNA interference, it was examined whether miR-101 overexpression and EZH2 knockdown generated similar gene expression profiles. To assess this, gene expression array analysis of SKBr3 cells transfected with either miR-101 or EZH2 siRNA duplexes was performed. Genes that were overexpressed at the 2-fold threshold were significantly overlapping in both the miR-101 and EZH2 siRNA transfected cells (=6.08e-17) (FIG. 16). Similarly, those genes that were repressed also had significant overlap (P=3.24e-27).

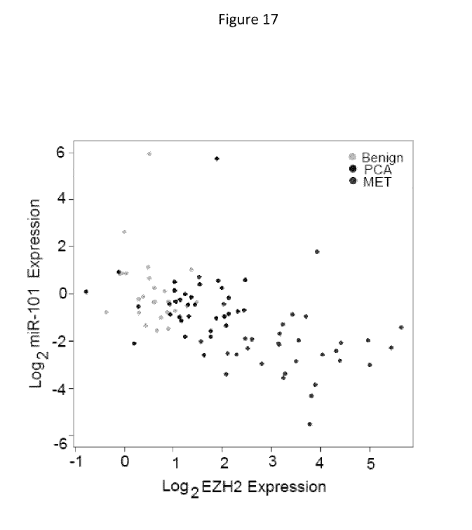

It was next investigated whether miR-101 expression inversely correlates with EZH2 levels in human tumors. A meta-analysis of a majority of the publicly available miRNA expression datasets suggested that miR-101 is significantly under-expressed in prostate, breast, ovarian, lung and colon cancers (Table 3). As EZH2 was initially found to be overexpressed in a subset of aggressive clinically localized prostate cancers and almost universally elevated in metastatic disease (Varambally et al., supra), miR-101 was examined in a similar context of prostate cancer progression by qPCR analysis (FIG. 4A, FIG. 17). Metastatic prostate cancers expressed significantly higher levels of EZH2 as compared to clinically localized disease or benign adjacent prostate tissue (P<0.0001). Consistent with a functional connection between miR-101 and EZH2, miR-101 expression was significantly decreased in metastatic prostate cancer relative to clinically localized disease or benign adjacent prostate tissue (P<0.0001). miR-217, which like miR-101 was predicted to regulate EZH2, did not exhibit significant differences between metastatic disease and clinically localized prostate cancer or benign prostate (P=0.35 and 0.13, respectively).

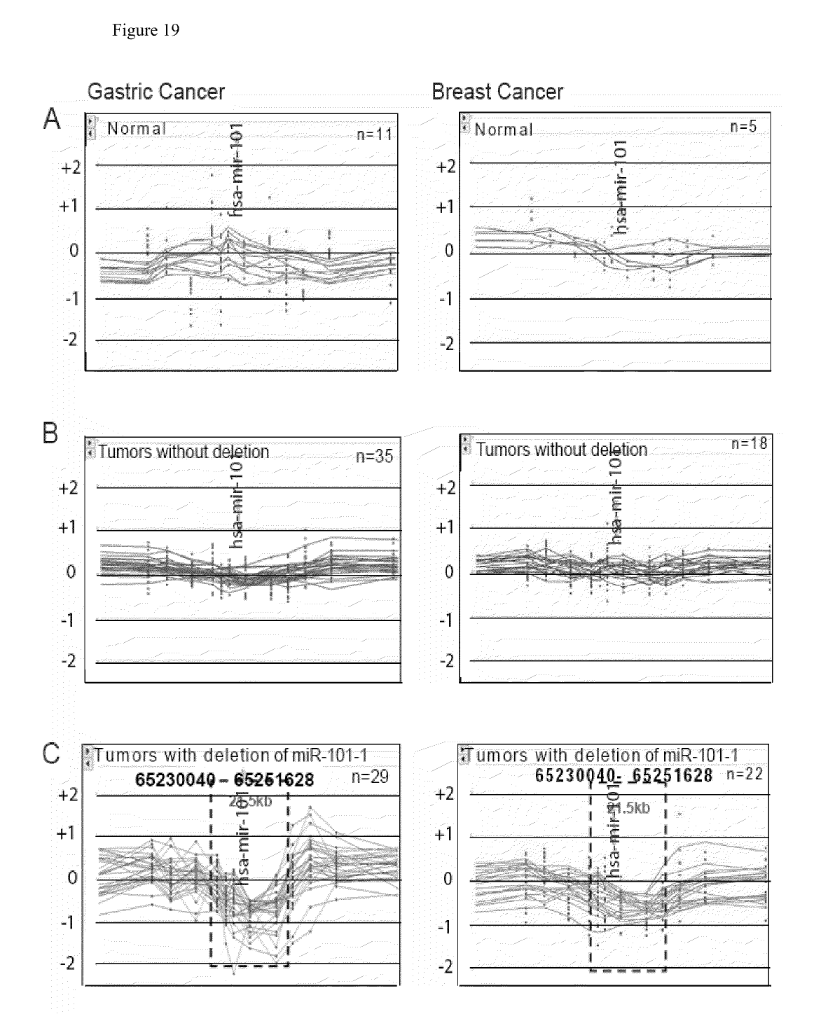

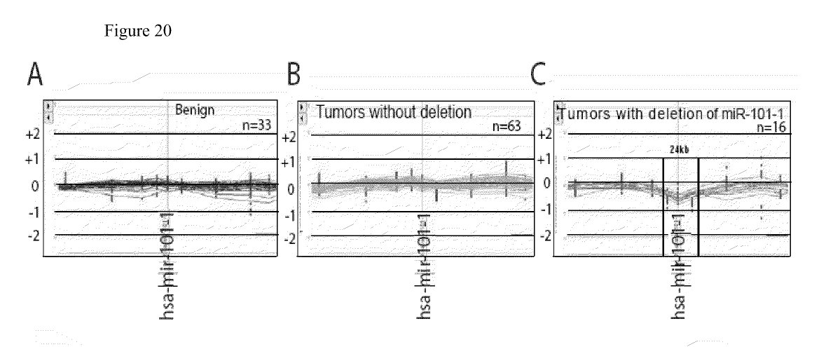

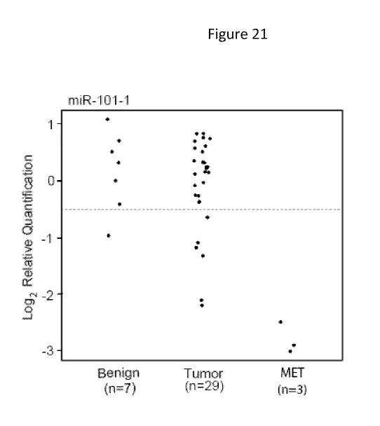

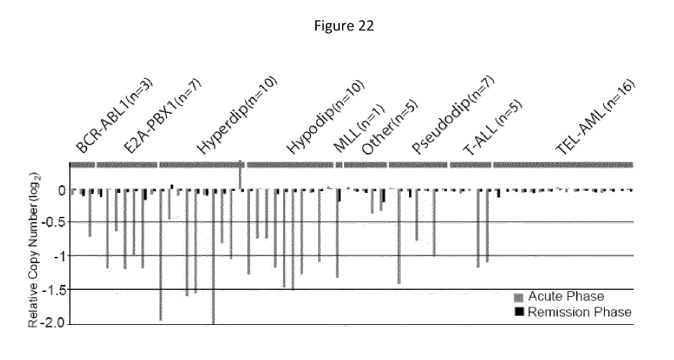

To extend the findings of miR-101 deletion to other solid tumors, breast cancers were examined. It was found that 6 of 29 breast cancers exhibited loss of miR-101-1 (FIG. 21). A single breast cancer metastasis was assayed. All 3 samples from this patient had marked copy number loss in the miR-101-1 locus. Genomic PCR of the miR-101-2 locus did not produce a significant number of breast cancers exhibiting loss. As EZH2 has been shown to be elevated in a wide variety of cancers (Plath et al., Science 300, 131 (2003), Kleer et al., supra; Takeshita et al., Proc. Natl. Acad. Sci. U.S.A. 102, 12177 (2005); Bachmann et al., J. Clin. Oncol. 24, 268 (2006); Weikert et al., Int. J. Mol. Med. 16, 349 (2005); Matsukawa et al., Cancer Sci. 97, 484 (2006); Lewis et al., Cell 120, 15 (2005); Marson et al., Cell 134, 521 (2008)), it was examined whether miR-101 genomic loss was similarly prevalent across cancer types. Agilent array CGH data for the region flanking the miR-101 from prostate, breast, and gastric cancer were compiled ((Tables 8-10 and FIGS. 19-20). In the prostate cancer Agilent array CGH dataset 22.7% (18/79) tumors exhibited complete loss or loss of heterozygosity of the miR-101 locus and 48.5% (16/33) of metastatic disease showed genomic loss of miR-101. Agilent breast cancer array CGH dataset (n=40), showed 60% of cancers with miR-101 loss, either complete deletion of loss of heterozygosity. Gastric cancer array CGH data indicated 56.2% (36/64) cases with miR-101 loss. Published data and publicly available data from The Cancer Genome Anatomy (TCGA) project was also interogated. Examining the glioblastoma multiforme dataset (n=145) from the TCGA it was observed that 18.7% of cases exhibit loss of miR-101. In a lung adenocarcinoma dataset (Cao et al., Oncogene (2008)) by Zhao et al (n=63), 37.3% of the cases exhibited loss of the miR-101. One of the most extensive studies represented in the meta-analysis (based on genomic coverage and number of patients) was by Mullighan et al, (Yu et al., Cancer Cell 12, 419 (2007)) which used 500K Affymetrix SNP arrays to study aberrations in 369 acute lymphoblastic leukemias (ALL) and controls. Of these, 127 were matched samples representing the active phase of disease (cancer) and remission phase (“normal”). 15.3% of the active phase samples exhibited loss of the miR-101-2 locus (and only 1.2% exhibited loss of miR-101-1) (FIG. 22). None of the matched controls (remission phase samples) displayed loss in either of the genomic loci confirming the somatic nature of this aberration. Furthermore, miR-101-2 loss does not occur frequently in the TEL-AML sub-type of ALL (FIG. 22).

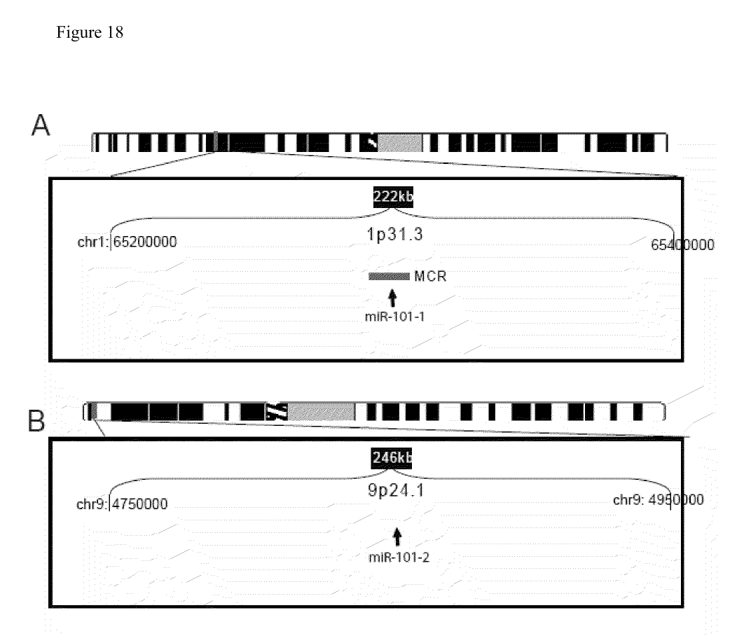

To investigate the mechanism for miR-101 transcript loss in prostate cancer progression, quantitative genomic PCR was performed for miR-101. Of note, miR-101 has two genomic loci that are on chromosome 1 (miR-101-1) and chromosome 9 (miR-101-2) (FIG. 18, A and B). Based on genomic PCR, 2 of 16 clinically localized prostate cancers and 17 of 33 metastatic prostate cancers exhibited loss of the miR-101-1 locus (FIG. 4B). Similarly, 4 of 16 clinically localized prostate cancers and 8 of 33 metastatic prostate cancers displayed loss of miR-101-2 (FIG. 4B). FIG. 4C displays a heatmap representation of matched samples in which miR-101 transcript, EZH2 transcript, miR-101-1 genomic loci and miR-101-2 genomic loci were monitored. EZH2 transcript levels were inversely associated with miR-101 transcript levels across prostate cancer progression to metastasis (P<0.0001). EZH2 tended to be uniformly elevated in samples in which the miR-101-1 or miR-101-2 genomic loci exhibited copy number loss (P=0.004, permutation test).

To formally demonstrate that genomic loss of miR-101 loci was somatic in nature, 9 metastatic prostate cancers that exhibited loss of miR-101-1 were identified and DNA was obtained from matched normal tissue. Eight of 9 cases exhibited a marked decrease in relative levels of miR-101-1 copy number in the cancer when compared to matched normal tissue (FIG. 4D). miR-101 genomic loss was investigated in other tumor types. Using a number of experimental platforms focal loss (˜20 kB) of miR-101-1 was demonstrated in a subset of breast, gastric and prostate cancers (FIGS. 19 to 21). Furthermore, public domain high-density array CGH and SNP array datasets were investigated and genomic loss of one or both miR-101 loci in a subset of glioblastoma multiforme, lung adenocarcinoma, and acute lymphocytic leukemia was identified (FIG. 21).

miR-101, by virtue of its regulation of EZH2, may have profound control over the epigenetic pathways active not only in cancer cells, but in normal pluripotent embryonic stem cells. Overexpression of miR-101 may configure the histone code of cancer cells to that associated with a more benign cellular phenotype. As the loss of miR-101 and concomitant elevation of EZH2 is most pronounced in metastatic cancer, it was contemplated that miR-101 loss may represent a progressive molecular lesion in the development of more aggressive disease.

In conclusion, this study show that experimentally only miR-101 directly regulates EZH2 expression and is inversely associated with EZH2 levels in cancer progression. miR-101 has properties consistent with that of a tumor suppressor gene that functions by negatively regulating the oncogenic potential of EZH2. Furthermore, miR-101, by virtue of its regulation of EZH2, controls epigenetic pathways active not only in cancer cells, but in normal pluripotent embryonic stem cells. Overexpression of miR-101 decreases the level of trimethyled histone H3K27 in cancer cells and configures the histone code of cancer cells to more of a benign phenotype. miR-101 is a functional regulator of the PRC2 complex and is expressed at high levels in differentiated cells and at low levels in embryonic stem cells and subsets of more aggressive cancers leading to EZH2 and PRC2 induction.

The present invention is not limited to a particular mechanism. Indeed, an understanding of the mechanism is not necessary to practice the present invention. Nonetheless, it is contemplated that the genetic mechanism for EZH2 elevation in cancer involves the somatic loss of the miR-101 genomic loci. Similar to EZH2 being broadly elevated in subsets of cancers, the meta-analysis revealed that the miR-101 genomic loci is lost in a subset of different cancer types. As the loss of miR-101 and concomitant elevation of EZH2 is most pronounced in metastatic cancer, it was contemplated that miR-101 loss represents a progressive molecular lesion in the development of more aggressive disease.

TABLE 1

| miRNA | MicroInspector | PicTar | TargetScan | miRanda |
|---|---|---|---|---|
| hsa-miR-101 | 1 | 1(2) | 2(2) | 1(2) |
| hsa-miR-151 | 2 | | | |
| has-miR-124a | | 3(2) | 6 | 8 |
| hsa-miR-17-3p | 3 | | | |
| has-miR-126* | | 19 | | |
| has-miR-138 | | 6(2) | 4 | 5 |
| has-miR-144 | | 4(2) | | |
| hsa-miR-181a | 4 | | | |
| hsa-miR-181b | 5 | | | |
| hsa-miR-185 | 6(2) | 18 | | 9 |
| has-miR-199a* | | 5(2) | | |
| has-miR-20a | 7 | 18 | | |
| has-miR-20b | | 13 | | |
| hsa-miR-214 | 8 | | | 9 |
| hsa-miR-217 | 9 | 2(2) | 1(2) | 3 |
| hsa-miR-25 | 10 | | 5 | |
| has-miR-26a | | 17 | 3 | |
| has-miR-26b | | 16 | 3 | |
| has-miR-32 | | 15 | 5 | |
| hsa-miR-324-3p | 11 | | | |
| hsa-miR-330 | 12(2) | | | 10 |
| has-miR-363 | | | 5 | |
| has-miR367 | | | 5 | |
| has-miR-506 | | | 7 | |
| has-let-7b | | 8 | 8 | 4 |
| has-let-7i | | 9 | 8 | 6 |
| has-let-7c | | 10 | 8 | 4 |
| has-let-7g | | 10 | 8 | 6 |
| has-let-7d | | 11 | 8 | 4 |
| has-let-7e | | 12 | 8 | 6 |
| has-let-7f | | 12 | 8 | 2 |
| has-let-7a | | 12 | 8 | 4 |
| has-miR-92 | | 14 | 5 | |
| has-miR-98 | | 7 | 8 | 7 |

TABLE 2

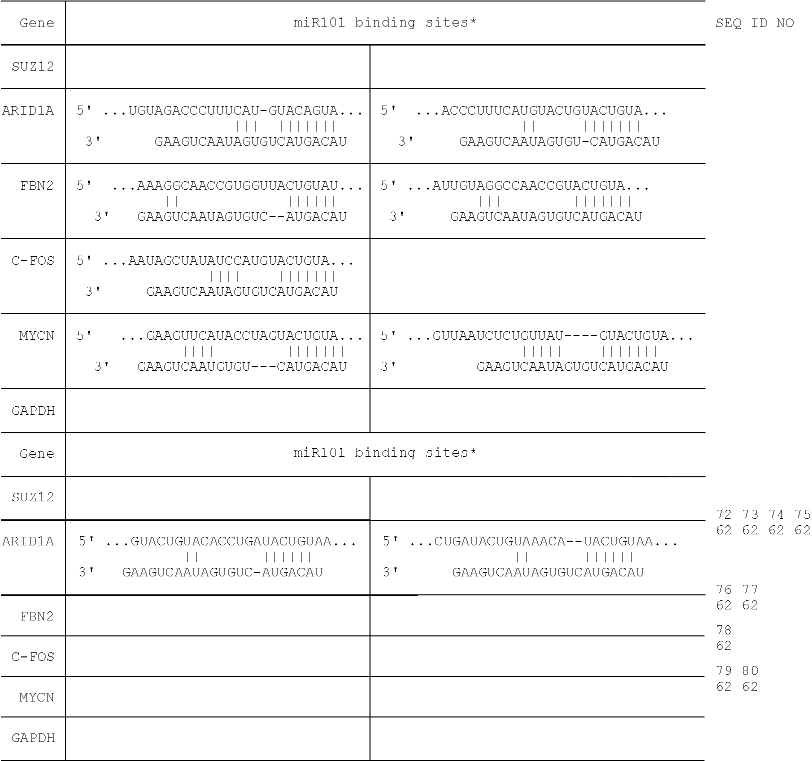

*Predicted consequential pairing of target region (top) and miRNA (bottom)

TABLE 3

| Cancer Type | First Autho [symbol] | Citation | Pubmed ID | Cancer(n) | Normal(n) | Expression | Percentile |
|---|---|---|---|---|---|---|---|
| Breast | Iorio | Cancer Research 2005; 65(16): 7065-70 | 16461460 | 76 | 10 | Down | 5th |
| Lung | Yanaihara | Cancer Cell 2006; 9(3): 189-98 | 16530703 | 104 | 104 | Down | 11th |
| Ovarian | Iorio | Cancer Research 2007; 67(18): 8699-707 | 17875710 | 74 | 15 | Down | 7th |
| Thyroid | Visone | Oncogene 2007; 26(54): 7590-5 | 17563749 | 10 | 10 | — | — |
| Prostate | Porkka | Cancer Resarch 2007; 67(13): 6130-5 | 17616669 | 9 | 4 | — | — |
| Colon | Schepeler | Cancer Research 2008; 68(15): 6416-24 | 13676867 | 49 | 10 | Down | 7th |
| All | Lu | Nature 2005; 435(7043): 834-8 | 15944708 | 140 | 46 | Down | 2nd |

[symbol]

indicates data missing or illegible when filed

TABLE 4

| Gene name | Forward primer | Reverse primer | SEQ ID NO | |
|---|---|---|---|---|
| EZH2 | TGCAGTTGCTTCAGTACCCATAAT | ATCCCCGTGTACTTTCCCATCATAAT | 5 | 6 |
| ADRB2 | TTCCTCTTTGCATGGAATTTG | AGAGGAGTGGGGGAAGAGTC | 7 | 8 |
| hDAB2IP | TGGACGATGTGCTCTATGCC | GGATGGTGATGGTTTGGTAG | 9 | 10 |
| RUNX3 | TCTGTAAGGCCCAAAGTGGGTA | ACCTCAGCATGACAATATGTCACAA | 11 | 12 |
| CIITA | CCGACACAGACACCATCAAC | CTTTTCTGCCCAACTTCTGC | 13 | 14 |
| CDH1 | GGAGGAGAGCGGTGGTCAAA | TGTGCAGCTGGCTCAAGTCAA | 15 | 16 |
| GAPDH | TGCACCACCAACTGCTTAGC | GGCATGGACTGTGGTCATGAG | 17 | 18 |

TABLE 5

| | genomic | |
|---|---|---|
| Gene location | region | RQ |
| chr1: 65,296,708-65,296,562 | miR101-1 | 1.14 |
| chr2: 56,063,616-56,063,502 | miR217 | 1.37 |
| chrX: 45490429-45490738 | miR221 | 0.46 |
| chrX: 45491265-45491574 | miR222 | 0.56 |
| chrX: 133508210-133508507 | miR424 | 0.56 |
| chrX: 113964173-113964483 | miR448 | 0.63 |
| chrX: 133507924-133508194 | miR503 | 0.67 |
| chrX: 137577438-137577720 | miR504 | 0.56 |
| chrX: 118664629-118664939 | miR766 | 0.66 |
| chrX: 77247072-77247271 | PGK1 | 0.54 |
| chrX: 77247822-77248021 | PGK2 | 0.48 |
| chrX: 77248872-77249071 | PGK3 | 0.55 |
| chr6: 170720579-170720690 | TBP | 1 |

TABLE 6

| | | | SEQ ID NO | |
|---|---|---|---|---|
| miR101-1 | GTACTGTGATAACTGAAGGATG | ATTCTGCTTCTCTTTGCCTTGT | 19 | 20 |
| miR101-2 | GACTGAACTGTCCTTTTTCGG | CCTTTCTCAATGTGATGGCA | 21 | 22 |
| miR217 | CTAATGCATTGCCTTCAGCA | TTAGCATCTTGGGCTCACCT | 23 | 24 |
| miR424 | ACCTGGTGGCAGGAACAC | TGAGGCGCTGCTATACCC | 25 | 26 |
| miR503 | CAGGCGATGGCCTAAGACT | CAGGGTAAGTCTGGGACTGC | 27 | 28 |
| miR766 | TGAAGACTCTGGGGACTTTTG | AATATACACAGAGGATTGCTTAGCC | 29 | 30 |
| miR448 | TGGCTGGTTGCATATGTAGG | TGGTGTTTCTGGTGTCTGTCA | 31 | 32 |
| miR384 | AAAACAAATGTTGCAATCCAAA | TGCAAATAACAAGATGCCTGA | 33 | 34 |
| miR222 | ACTGAGCCATTGAGGGTACCTA | CCCCAGAAGGCAAAGGAT | 35 | 36 |
| miR221 | GTGAGACAGCCAATGGAGAAC | TGTTCGTTAGGCAACAGCTACA | 37 | 38 |
| miR934 | CAGCCTTTGATGGTGTGTGT | TCCATTACTGGAGACTCTGGG | 39 | 40 |
| TBP | TTAGCTGGCTCTGAGTATGAATAAC | GCTGGAAAACCCAACTTCTG | 41 | 42 |

TABLE 7

| Gene name | Forward primer | Reverse primer | SEQ ID NO | |
|---|---|---|---|---|
| ADRB2 | GTGACTTTATGCCCCTTTAGAGACAA | GAAGGGCTACAACTGGAACTGGAATA | 43 | 44 |
| DAB2IP | ATTCCTCCAGGTGGGTGTGG | CTAAGCCGCTGTTGCCTTGGC | 45 | 46 |
| CIITA | TCCTGGCCCGGGGCCTGG | CTGTTCCCCGGGCTCCCGC | 47 | 48 |
| RUNX3 | TGTCCCGGGATCCTCTTCT | TAGAGACGTTGGTGCGGAAAT | 49 | 50 |
| CDH1 | TAGAGGGTCACCGCGTCTAT | TCACAGGTGCTTTGCAGTTC | 51 | 52 |
| WNT1 | GTTTCTGCTACGCTGCTGCT | CACCAGCTCACTTACCACCA | 53 | 54 |
| GAPDH | TACTAGCGGTTTTACGGGCG | TCGAACAGGAGGAGCAGAGAGCGA | 55 | 56 |
| KIAA0066 | CTAGGAGGGTGGAGGTAGGG | GCCCCAAACAGGAGTAATGA | 57 | 58 |
| NUP214 | CAGTGAGGTCTCAGCATCAGCA | CTGGAGGCTATGGGGGTACTTG | 59 | 60 |

TABLE 8

| A | B | C | D | E | F |
|---|---|---|---|---|---|
| gene | hsa-mir-101-1 | hsa-mir-101-1 | hsa-mir-217 | hsa-mir-101-2 | hsa-mir-101-2 |
| probe | A_16_P15150840 | A_16_P15150873 | A_16_P35687642 | A_16_P02051568 | A_16_P18537000 |
| chromosome | chr1:065227167-224 | chr1:065237759-818 | chr2:056060746-805 | ch9:004831297-356 | chr9:004839536-595 |
| CAP_M_1 | −0.0809 | −0.0809 | −0.0341 | −0.2261 | −0.2261 |
| CAP_M_10 | −0.7481 | −0.7481 | 0.216 | −0.0775 | −0.0775 |
| CAP_M_11 | 0.0254 | 0.0254 | 0.0284 | 0.0112 | 0.0112 |
| CAP_M_12 | −0.2665 | −0.2665 | 0.4022 | 0.2973 | 0.2973 |
| CAP_M_13 | −0.0141 | −0.0141 | 0.2593 | 0.0282 | 0.0282 |
| CAP_M_14 | −1.8143 | −1.8143 | −0.1059 | 0.0951 | 0.0951 |
| CAP_M_15 | −1.227 | −1.227 | 0.1283 | −0.1659 | −0.1659 |
| CAP_M_16 | 0.1715 | 0.1715 | 0.2321 | −0.5019 | −0.5019 |
| CAP_M_17 | 0.0705 | 0.0705 | 0.24 | −0.8308 | −0.8308 |
| CAP_M_18 | 0.8552 | 0.8552 | −1.3471 | 0.0101 | 0.0101 |
| CAP_M_19 | 0.0659 | 0.0659 | 0.5654 | 0.4472 | 0.4472 |
| CAP_M_2 | −0.0776 | −0.0776 | −0.0611 | −0.0833 | −0.0883 |
| CAP_M_20 | −0.0085 | −0.0085 | −0.5042 | 0.7471 | 0.7471 |
| CAP_M_21 | 0.1826 | 0.1826 | −0.0842 | −0.0834 | −0.0834 |
| CAP_M_22 | 0.1887 | 0.1887 | 0.2508 | 0.6486 | 0.6486 |
| CAP_M_23 | −0.4859 | −0.4859 | 0.1719 | −0.5443 | −0.5443 |
| CAP_M_24 | 0.0769 | 0.0769 | 0.2559 | 0.6235 | 0.6235 |
| CAP_M_26 | −0.0956 | −0.0956 | −0.1266 | −0.1389 | −0.1389 |
| CAP_M_27 | 0.0822 | 0.0822 | 0.5059 | 0.0579 | 0.0579 |
| CAP_M_28 | −0.0821 | −0.0821 | −0.1024 | 0.3122 | 0.3122 |
| CAP_M_29 | −0.0525 | −0.0525 | −5.00E−04 | −1.5964 | −1.5964 |
| CAP_M_3 | 0.0193 | 0.0193 | −0.0267 | 0.3568 | 0.3568 |
| CAP_M_30 | −0.1362 | −0.1362 | 0.0664 | −0.2614 | −0.2614 |
| CAP_M_31 | −0.1079 | −0.1079 | −0.1385 | −0.102 | −0.102 |
| CAP_M_33 | −0.0651 | −0.0651 | −0.0839 | −0.1168 | −0.1168 |
| CAP_M_34 | −1.0769 | −1.0769 | −0.0277 | −0.0223 | −0.0223 |
| CAP_M_36 | −0.0137 | −0.0137 | −0.5804 | −0.5466 | −0.5466 |
| CAP_M_37 | 0.0191 | 0.0191 | 0.0183 | 0.0017 | 0.0017 |
| CAP_M_38 | −0.0443 | −0.0443 | −0.0407 | 0.5421 | 0.5421 |
| CAP_M_39 | −0.2888 | −0.2888 | −0.0374 | 0.7081 | 0.7081 |
| CAP_M_4 | −0.0164 | −0.0164 | −0.046 | −0.3367 | −0.3367 |
| CAP_M_40 | −0.4486 | −0.4486 | 0.1381 | −0.1165 | −0.1165 |
| CAP_M_41 | −0.7118 | −0.7118 | −0.0902 | −0.1655 | −0.1655 |
| CAP_M_43 | −0.0754 | −0.0754 | 0.1981 | −0.1125 | −0.1125 |
| CAP_M_44 | −0.0795 | −0.0795 | −0.0492 | −0.0952 | −0.0952 |
| CAP_M_5 | 0.2263 | 0.2263 | −0.3897 | −0.1157 | −0.1157 |
| CAP_M_6 | −0.0604 | −0.0604 | −0.06 | −0.6029 | −0.6029 |
| CAP_M_7 | −0.3393 | −0.3393 | −0.2452 | 0.059 | 0.059 |
| CAP_M_8 | −1.2465 | −1.2465 | −0.0276 | −0.039 | −0.039 |
| CAP_M_9 | 0.1546 | 0.1546 | 0.0894 | 0.1187 | 0.1187 |
| CAP_N_1 | 0.0292 | 0.0292 | −0.0197 | −0.0081 | −0.0081 |
| CAP_N_10 | 0.0077 | 0.0077 | −0.0087 | −0.0043 | −0.0043 |
| CAP_N_11 | −0.0026 | −0.0026 | −0.003 | −0.0113 | −0.0113 |
| CAP_N_12 | −0.0024 | −0.0024 | −0.0044 | −0.0054 | −0.0054 |
| CAP_N_13 | 0.0445 | 0.0445 | 0.2069 | −0.1313 | −0.1313 |
| CAP_N_14 | 0.0386 | 0.0386 | 0.0932 | −0.0876 | −0.0876 |
| CAP_N_15 | 0.0217 | 0.0217 | 0.0673 | −0.0161 | −0.0161 |
| CAP_N_16 | −0.0439 | −0.0439 | −0.0163 | −0.0246 | −0.0246 |
| CAP_N_17 | 0.0071 | 0.0071 | −0.0039 | −0.0041 | −0.0041 |
| CAP_N_18 | −0.3788 | −0.3788 | 0.2451 | 0.0546 | 0.0546 |
| CAP_N_19 | 0.0662 | 0.0662 | 0.008 | 0.0469 | 0.0469 |
| CAP_N_2 | −0.0021 | −0.0021 | −0.0104 | −0.0143 | −0.0143 |
| CAP_N_20 | −0.074 | −0.074 | −0.029 | −0.0432 | −0.0432 |
| CAP_N_21 | −0.0157 | −0.0157 | 0.009 | −0.0115 | −0.0115 |
| CAP_N_22 | 0.0105 | 0.0105 | 0.011 | −0.009 | −0.009 |
| CAP_N_23 | −0.0251 | −0.0251 | 0.0037 | 0.0022 | 0.0022 |
| CAP_N_24 | −0.0093 | −0.0093 | 0.005 | 0.0164 | 0.0164 |
| CAP_N_25 | 0.0548 | 0.0548 | 0.0156 | 0.0377 | 0.0377 |
| CAP_N_26 | 6.00E−04 | 6.00E−04 | 0.0016 | 0.014 | 0.014 |
| CAP_N_27 | −0.0247 | −0.0247 | −0.0086 | −0.0198 | −0.0198 |
| CAP_N_28 | −0.0164 | −0.0164 | −0.0413 | −0.0168 | −0.0168 |
| CAP_N_29 | −0.0306 | −0.0306 | −0.0222 | −0.0373 | −0.0373 |
| CAP_N_3 | −0.0385 | −0.0385 | −0.0157 | −0.037 | −0.037 |
| CAP_N_30 | −0.0157 | −0.0157 | −0.0163 | −0.0174 | −0.0174 |
| CAP_N_31 | −0.0322 | −0.0322 | −0.0458 | −0.0559 | −0.0559 |
| CAP_N_32 | −0.0379 | −0.0379 | −0.0409 | −0.037 | −0.037 |
| CAP_N_33 | −0.0449 | −0.0449 | −0.0446 | −0.0344 | −0.0344 |
| CAP_N_4 | −0.0529 | −0.0529 | −0.0172 | −0.0183 | −0.0183 |
| CAP_N_5 | −0.0572 | −0.0572 | −0.0155 | −0.0514 | −0.0514 |
| CAP_N_6 | −0.2773 | −0.2773 | 0.2397 | −0.0202 | −0.0202 |
| CAP_N_7 | −0.2018 | −0.2018 | 0.2087 | −0.0108 | −0.0108 |
| CAP_N_8 | 0.0197 | 0.0197 | −0.0054 | −0.0158 | −0.0158 |
| CAP_N_9 | −0.0388 | −0.0388 | −0.0045 | −0.0385 | −0.0185 |
| CAP_T_1 | −0.033 | −0.033 | −0.265 | −0.0179 | −0.0179 |
| CAP_T_10 | 0.0028 | 0.0028 | 0.0079 | 0.0078 | 0.0078 |
| CAP_T_11 | −0.0648 | −0.0648 | −0.0331 | −0.0468 | −0.0468 |
| CAP_T_12 | −0.0225 | −0.0225 | −0.0225 | −0.0313 | −0.0313 |
| CAP_T_17 | 0.0303 | 0.0303 | −0.0172 | −0.0828 | −0.0828 |
| CAP_T_18 | −0.0031 | −0.0031 | 0.0401 | 0.124 | 0.124 |
| CAP_T_19 | −0.0254 | −0.0254 | −0.0076 | −0.0311 | −0.0311 |
| CAP_T_2 | −0.0243 | −0.0243 | −0.0203 | −0.0257 | −0.0257 |
| CAP_T_20 | −0.03 | −0.03 | −0.0197 | −0.0191 | −0.0191 |
| CAP_T_21 | −0.0237 | −0.0237 | −0.0065 | −0.0208 | −0.0208 |
| CAP_T_22 | −0.0154 | −0.0154 | −0.0027 | −0.0065 | −0.0065 |
| CAP_T_23 | −0.065 | −0.065 | −0.0311 | −0.1297 | −0.1297 |
| CAP_T_24 | 0.0493 | 0.0493 | −0.0275 | −2.00E−04 | −2.00E−04 |
| CAP_T_25 | −0.0399 | −0.0399 | −0.0245 | 0.0098 | −0.0098 |
| CAP_T_26 | 0.0332 | 0.0332 | 0.0064 | 0.0112 | 0.0112 |
| CAP_T_27 | 0.043 | 0.043 | 0.0587 | 0.0578 | 0.0578 |
| CAP_T_28 | 0.0093 | 0.0093 | 0.0062 | −0.0085 | −0.0085 |
| CAP_T_29 | 0.0272 | 0.0272 | 0.0077 | 0.0064 | 0.0064 |
| CAP_T_3 | −0.0038 | −0.0038 | −0.0032 | −0.0148 | −0.0148 |
| CAP_T_30 | 0.0052 | 0.0052 | −0.0071 | −0.012 | −0.012 |
| CAP_T_31 | 0.043 | 0.043 | 0.0112 | 0.0095 | 0.0095 |
| CAP_T_32 | 0.0327 | 0.0327 | 0.0104 | −0.0558 | −0.0558 |
| CAP_T_33 | 0.0821 | 0.0821 | 0.065 | 0.0837 | 0.0837 |
| CAP_T_34 | 0.0432 | 0.0432 | 0.0734 | 0.0637 | 0.0637 |
| CAP_T_35 | 0.0947 | 0.0947 | 0.586 | 0.0798 | 0.0798 |
| CAP_T_36 | −0.0273 | −0.0273 | −0.0307 | −0.1164 | −0.1164 |
| CAP_T_37 | −0.3564 | −0.3564 | −0.0041 | −0.0059 | −0.0059 |
| CAP_T_38 | 0.0698 | 0.0698 | 0.0516 | 0.0802 | 0.0802 |
| CAP_T_39 | 0.024 | 0.024 | 0.0072 | 0.0048 | 0.0048 |
| CAP_T_4 | −0.0289 | −0.0289 | −0.0237 | −0.0314 | −0.314 |
| CAP_T_40 | −0.0011 | −0.0011 | 0.0031 | −0.0658 | −0.0658 |
| CAP_T_41 | −0.0571 | −0.0571 | −0.0246 | −0.0198 | −0.0198 |
| CAP_T_42 | −0.0357 | −0.0357 | −0.0141 | −0.0233 | −0.0233 |
| CAP_T_43 | −0.0245 | −0.0245 | −0.005 | −0.0039 | −0.0039 |
| CAP_T_44 | 0.0221 | 0.0221 | 0.0186 | −0.0054 | −0.0054 |
| CAP_T_45 | 0.0245 | 0.0245 | 0.0323 | −0.0205 | −0.0205 |
| CAP_T_46 | −0.0117 | −0.0117 | 0.0055 | −0.0064 | −0.0064 |
| CAP_T_47 | −5.00E−04 | −5.00E−04 | −0.0317 | −9.00E−04 | −9.00E−04 |
| CAP_T_48 | 0.0075 | 0.0078 | 0.149 | −0.0052 | −0.O052 |
| CAP_T_49 | −0.0261 | −0.0261 | −0.0171 | −0.0402 | −0.0402 |
| CAP_T_5 | 0.0324 | 0.0324 | 0.0265 | 0.0197 | 0.0197 |
| CAP_T_50 | −0.0291 | −0.0291 | −0.0303 | −0.0232 | −0.0232 |
| CAP_T_51 | −0.0171 | −0.0171 | −0.0117 | −0.0127 | −0.0127 |
| CAP_T_52 | −0.0076 | −0.0076 | 3.00E−04 | −0.0031 | −0.0031 |
| CAP_T_53 | 0.0278 | 0.0278 | 0.0194 | 0.0282 | 0.0282 |
| CAP_T_54 | 0.0233 | 0.0233 | 0.0155 | 0.0089 | 0.0089 |
| CAP_T_55 | −0.0106 | −0.0106 | −0.0005 | 0.0134 | 0.0134 |
| CAP_T_56 | 0.0013 | 0.0013 | −3.00E−04 | −0.0172 | −0.0172 |
| CAP_T_57 | −0.0011 | −0.0011 | 0.2039 | −0.059 | −0.059 |
| CAP_T_58 | −0.0473 | −0.0473 | 0.0072 | −0.0138 | −0.0138 |
| CAP_T_59 | −0.0389 | −0.0389 | −0.0231 | 0.008 | 0.008 |
| CAP_T_6 | 0.0313 | 0.0313 | 0.0385 | 0.0315 | 0.0315 |
| CAP_T_60 | 0.0057 | 0.0057 | 0.003 | −0.0219 | −0.0219 |
| CAP_T_61 | 0.0517 | 0.0517 | −0.0352 | 0.0537 | 0.0537 |
| CAP_T_62 | 0.0185 | 0.0185 | 0.0248 | −0.5108 | −0.5108 |
| CAP_T_63 | 3.00E−04 | 3.00E−04 | −0.0016 | −0.0181 | −0.0181 |
| CAP_T_64 | −0.0027 | −0.0027 | −0.0065 | −0.0044 | −0.0044 |
| CAP_T_65 | 0.0128 | 0.0128 | 0.003 | −0.6371 | −0.6371 |
| CAP_T_66 | 0.0515 | 0.0515 | 0.0555 | −0.0058 | −0.0058 |
| CAP_T_67 | −0.0566 | −0.0566 | −0.0455 | 0.0588 | 0.00588 |
| CAP_T_68 | −0.0214 | −0.0214 | −0.0098 | −0.024 | −0.024 |
| CAP_T_69 | 0.0399 | 0.0399 | −0.0332 | −0.1052 | −0.1052 |
| CAP_T_7 | −0.0214 | −0.0214 | 0.0038 | −0.0064 | −0.0064 |
| CAP_T_70 | 0.0295 | 0.0295 | −0.0485 | −0.0018 | −0.0018 |
| CAP_T_71 | 0.0608 | 0.0608 | 0.0522 | 0.0534 | 0.0534 |
| CAP_T_72 | 0.0022 | 0.0022 | −0.0073 | −0.025 | −0.025 |
| CAP_T_73 | 0.0592 | 0.0592 | 0.1251 | −0.0162 | −0.0162 |
| CAP_T_74 | 0.0034 | 0.0034 | −0.0365 | −0.0558 | −0.0558 |
| CAP_T_8 | 0.0381 | 0.0361 | 0.2084 | −0.0257 | −0.0257 |
| CAP_T_9 | −0.0012 | −0.0012 | 0.0086 | 0.0053 | 0.0053 |

*Column B to F represents log 2 transformed Relative probe intensity compared to normal

TABLE 9

| [symbol] | B | C | [symbol] | [symbol] | [symbol] | [symbol] | [symbol] | [symbol] | [symbol] | [symbol] | [symbol] |
|---|---|---|---|---|---|---|---|---|---|---|---|
| gene | [symbol] | [symbol] | [symbol] | [symbol] | [symbol] | [symbol] | [symbol] | [symbol] | [symbol] | [symbol] | [symbol] |
| probe | [symbol] | [symbol] | [symbol] | [symbol] | [symbol] | [symbol] | [symbol] | [symbol] | [symbol] | [symbol] | [symbol] |
| chromosome | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |

*Column B to L represents log 2 transformed Relative probe intensity compared to normal [symbol]
indicates data missing or illegible when filed

TABLE 10

| [symbol] | B | [symbol] | [symbol] | E | [symbol] | [symbol] | [symbol] | [symbol] | [symbol] | [symbol] | [symbol] |
|---|---|---|---|---|---|---|---|---|---|---|---|
| gene | [symbol] | [symbol] | [symbol] | [symbol] | [symbol] | [symbol] | [symbol] | [symbol] | [symbol] | [symbol] | [symbol] |
| probe | [symbol] | [symbol] | [symbol] | [symbol] | [symbol] | [symbol] | [symbol] | [symbol] | [symbol] | [symbol] | [symbol] |
| chromosome | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] |

*Column B to L represents log 2 transformed Relative probe intensity compared to normal [symbol]
indicates data missing or illegible when filed

### Example 2

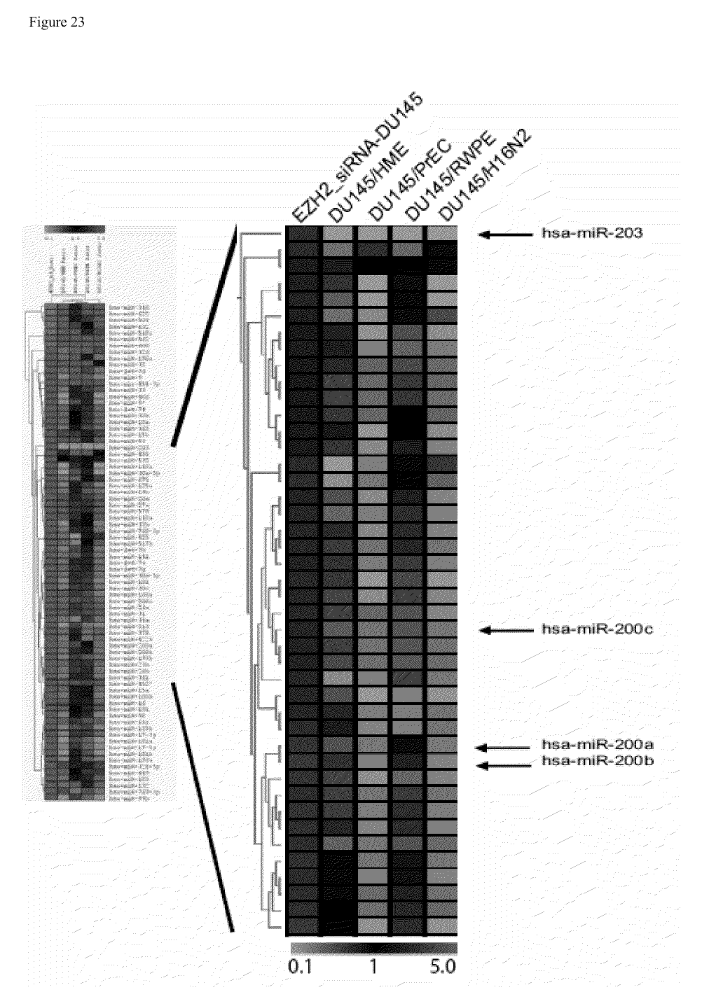

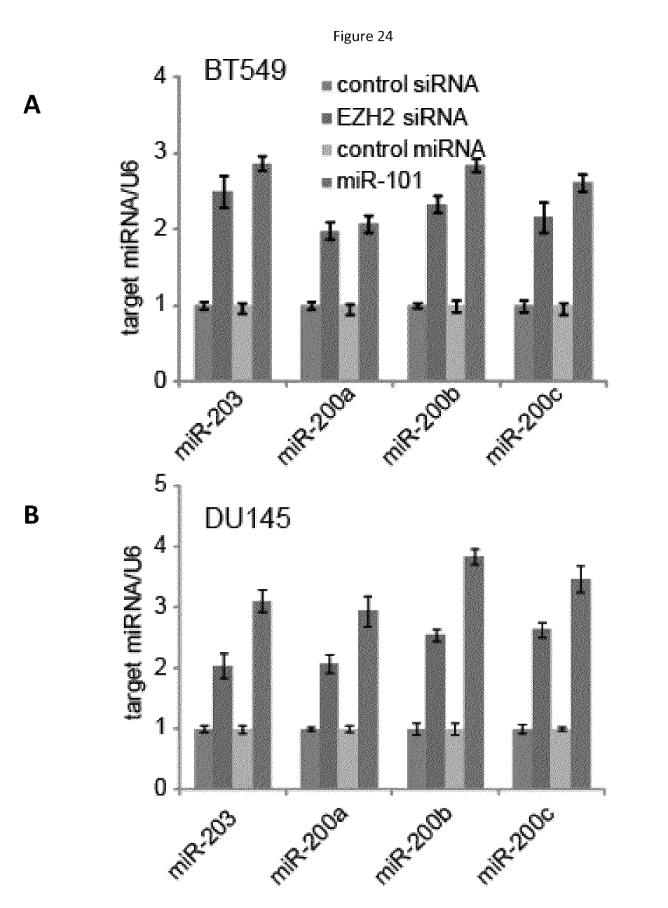

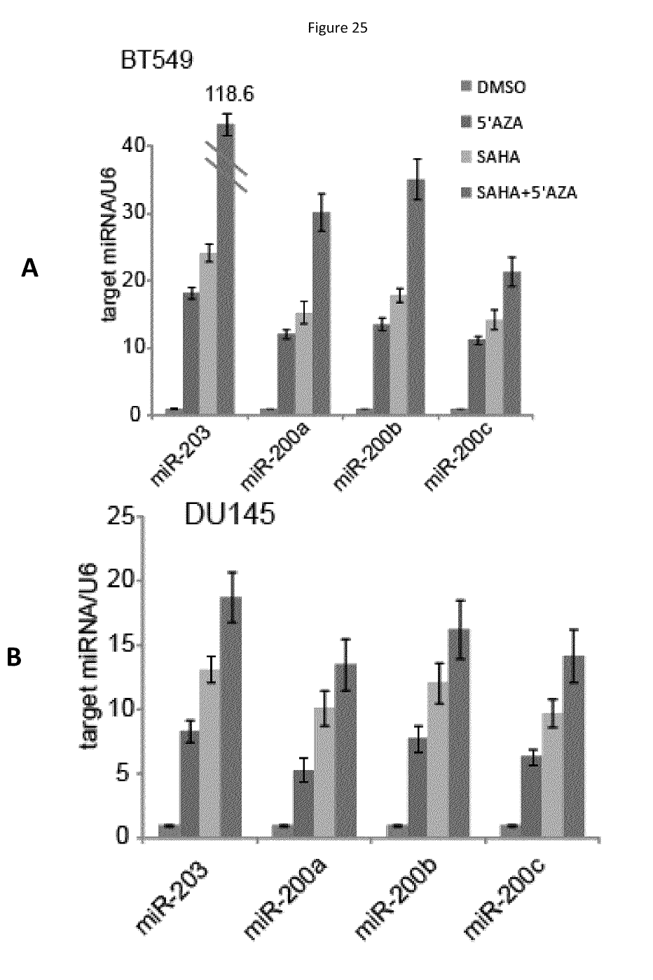

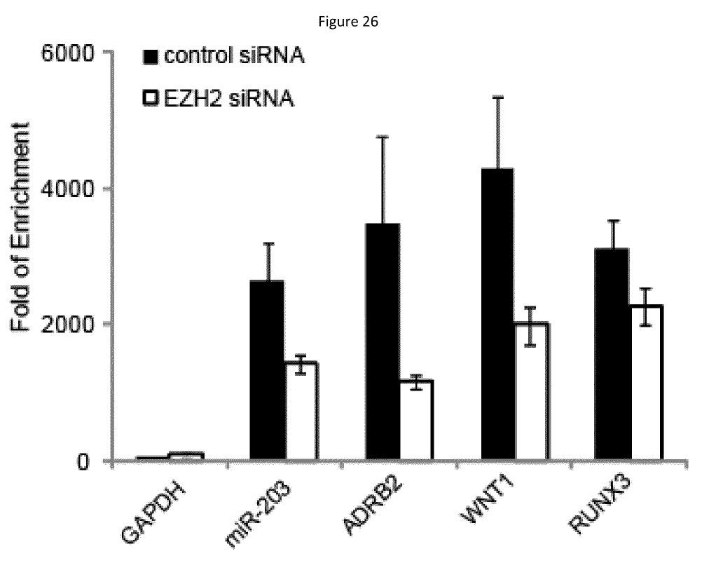

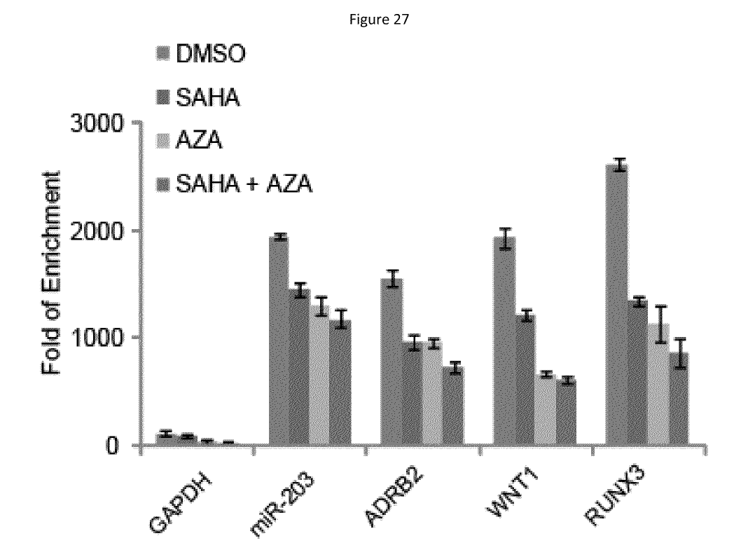

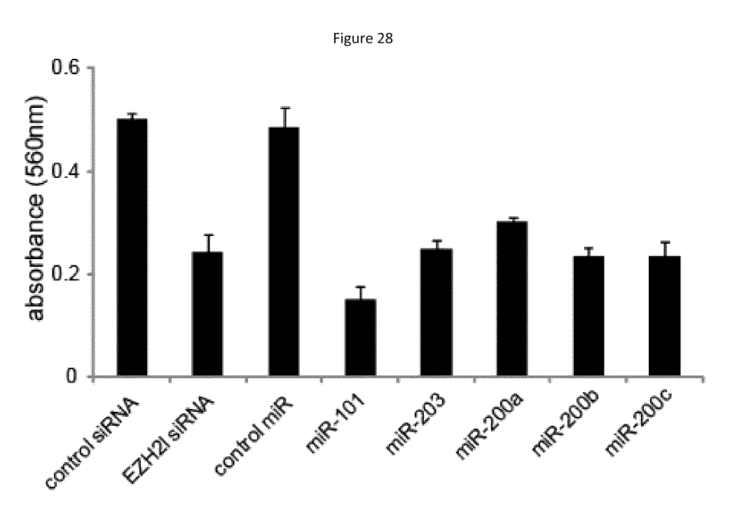

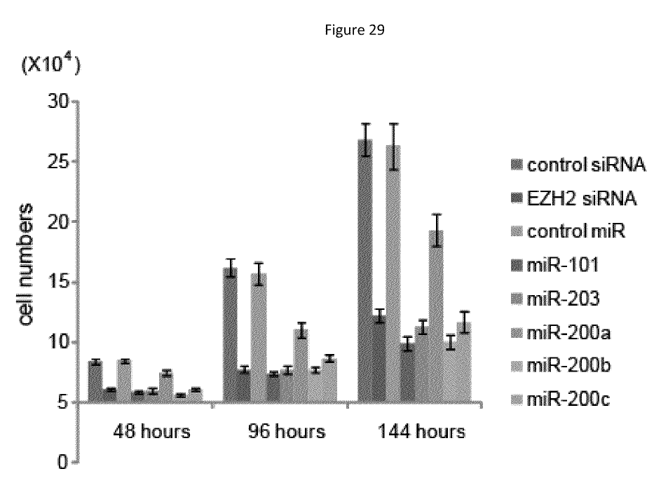

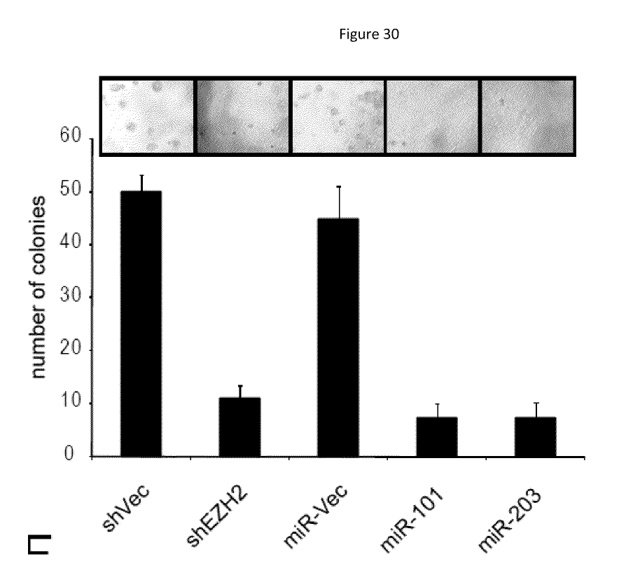

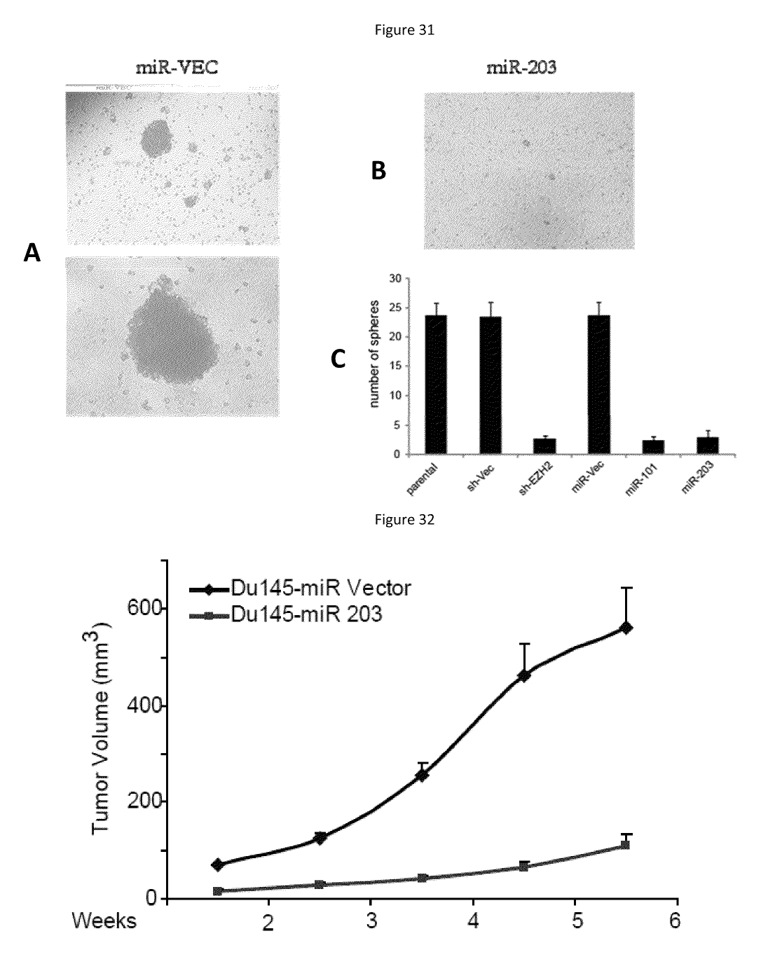

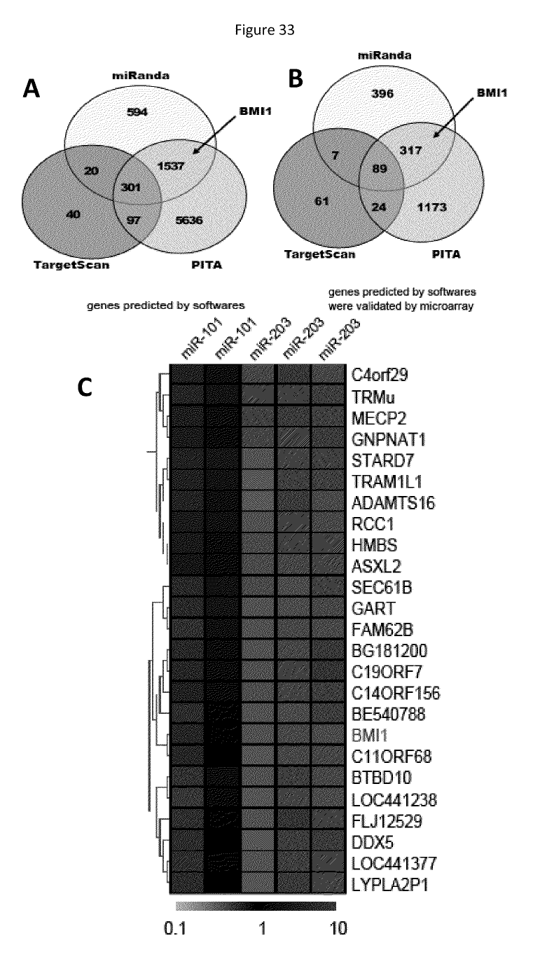

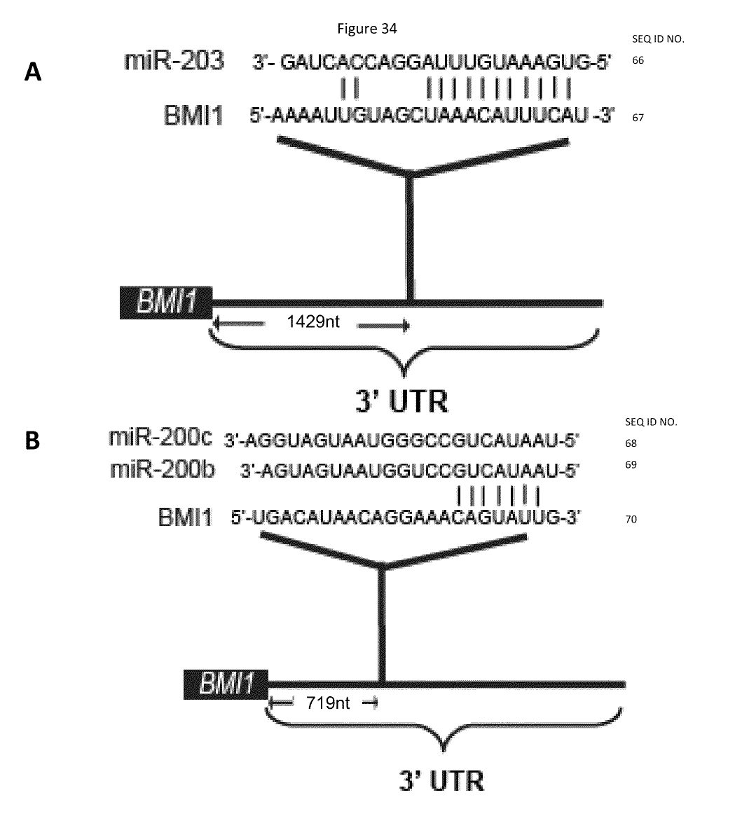

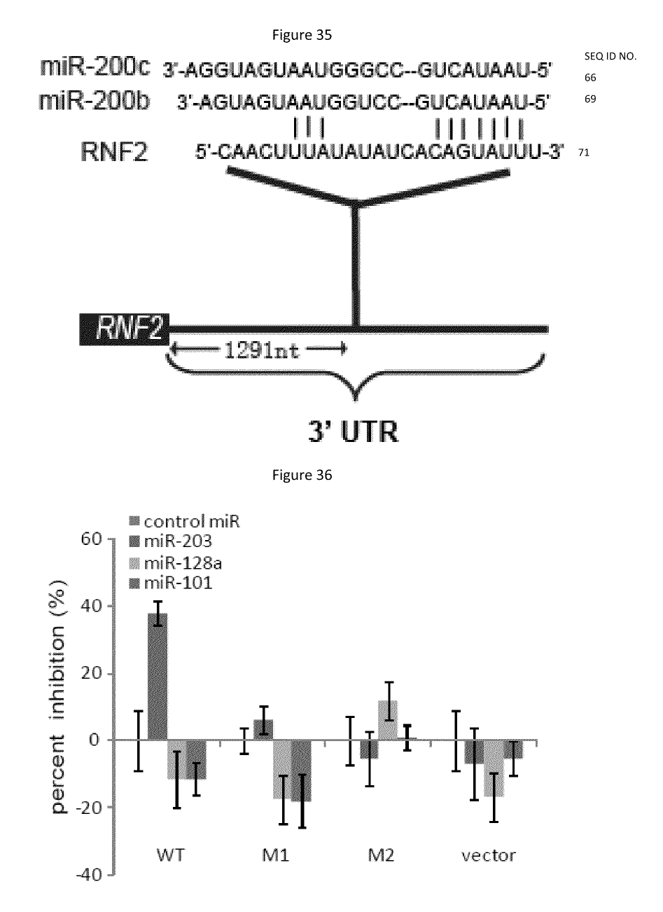

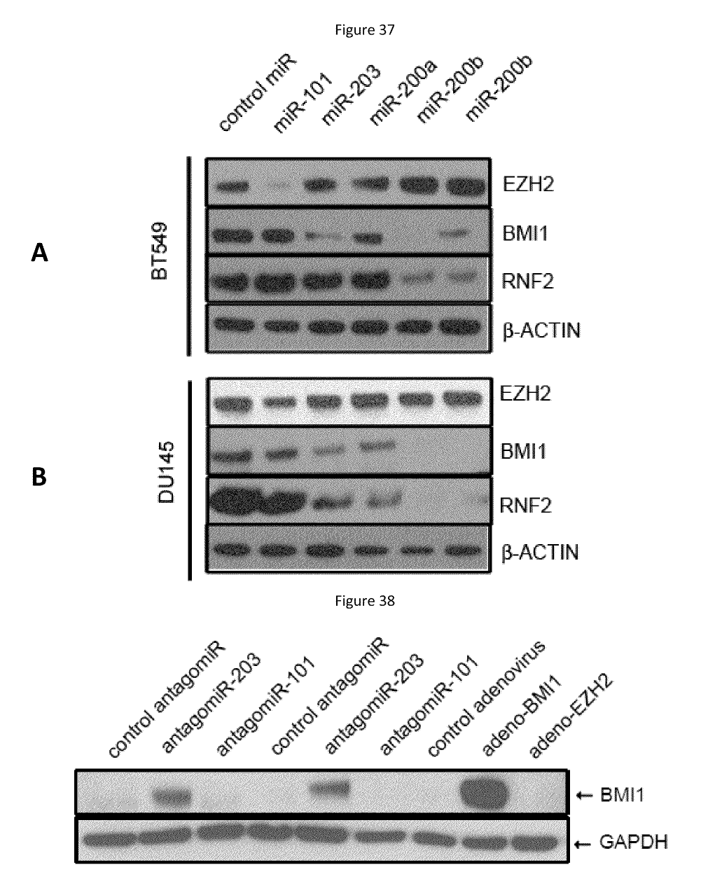

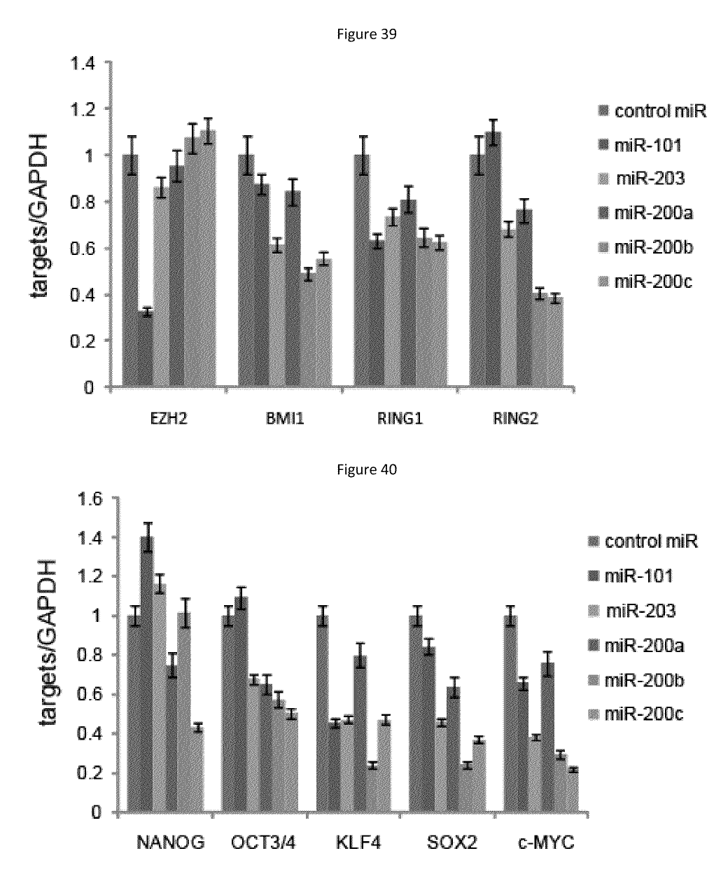

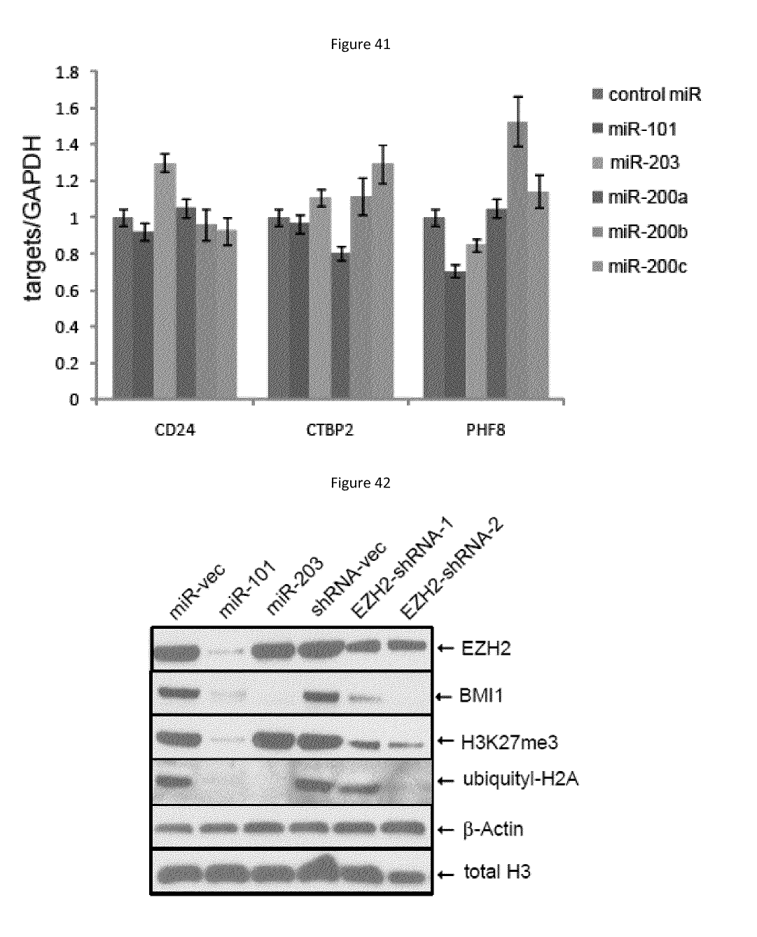

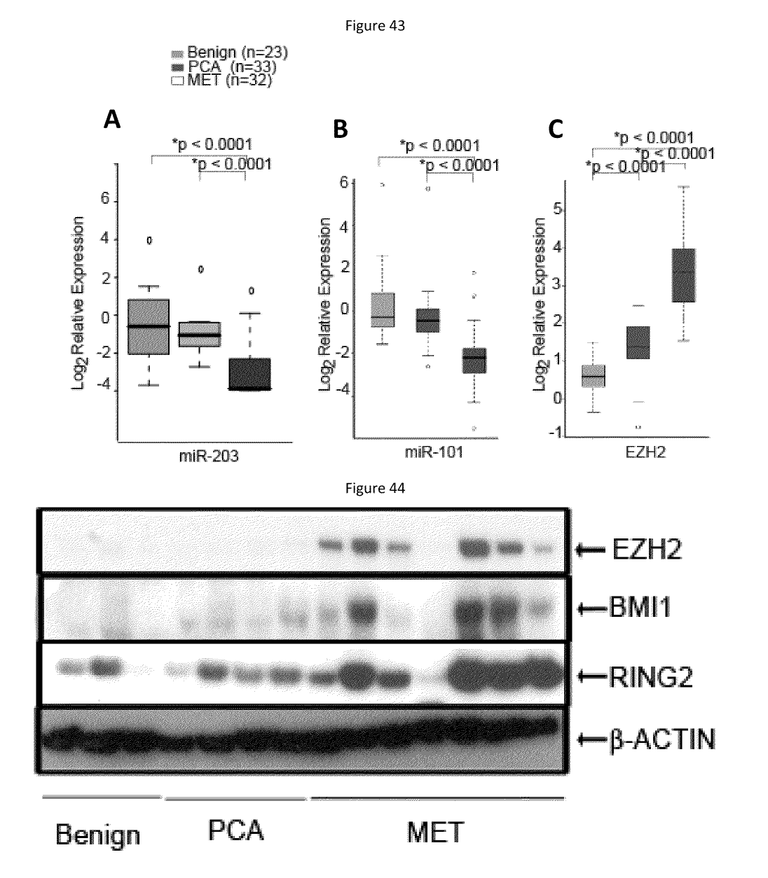

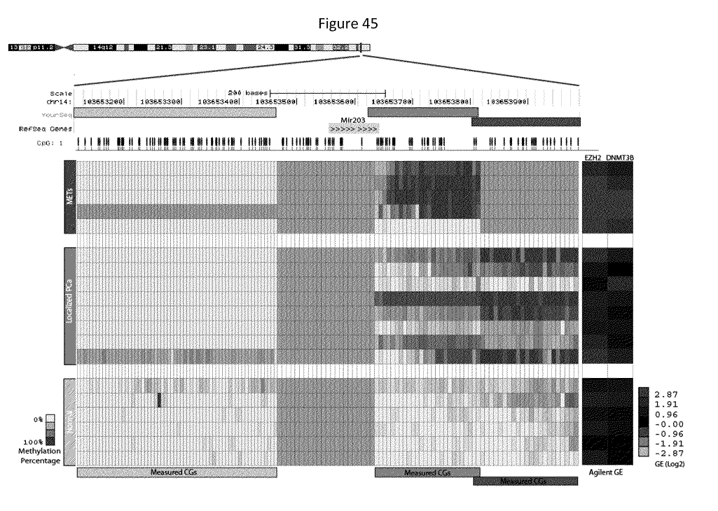

This Example describes a link between polycomb repressor complex 1 (PRC10 and 2 (PRC2) through miR-203 and miR-200 b,c. FIGS. 23-45 show that miR203 and miR200a,b,c are regulated by EZH2 and that miR-203 and miR200 inhibit cell invasion and growth. The Figures further demonstrate that BMI1 and RNF2 are predicted targets of miR-203 and miR200b,c and that mrR203 and miR-200 repress BMI1 and RNF2/RING2 protein levels. It was further shown that miR-203 is down regulated in metastatic prostate cancer. In addition, the miR-203 region is methylated in LnCAP (cancerous) but not PREC cells.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

We claim:

1. A method for identifying cancer in a patient comprising:
detecting the presence or absence in the sample of miR-101, wherein the absence of miR-101 in the sample identifies cancer in the patient.

2. The method of claim 1, wherein said cancer is prostate cancer.

3. The method of claim 1, wherein said cancer is selected from the group consisting of breast cancer, ovarian cancer, lung cancer gastric cancer, brain cancer, leukemia and colon cancer.

4. The method of claim 1, wherein said detecting comprises detecting the presence or absence of miR-101 RNA.

5. The method of claim 1, wherein said detecting comprises detecting the presence or absence of genomic DNA encoding miR-101.

6. The method of claim 1, further comprising the step of detecting the presence or absence of overexpression of EZH2 in said sample.

7. A method of inhibiting the growth of a cancer cell, comprising providing an exogenous nucleic acid encoding a miR-101 to a cancer cell under conditions such that the growth of said cancer cell is inhibited.

8. The method of claim 7, wherein said cancer cell is ex vivo.

9. The method of claim 7, wherein said cancer cell is in vitro.

10. The method of claim 7, wherein said cancer cell is in an animal.

11. The method of claim 10, wherein said animal is selected from the group consisting of a human and a non-human animal.

12. The method of claim 7, wherein said cancer is prostate cancer.

13. The method of claim 7, wherein said cancer is selected from the group consisting of breast cancer, ovarian cancer, lung cancer gastric cancer, brain cancer, leukemia and colon cancer.


SEQUENCE LISTING (80 sequences)

SEQ ID NO: 1 - DNA, 22 bp, Homo sapiens
tacagtactgtgataactgaag

SEQ ID NO: 2 - RNA, 75 nt, Homo sapiens
ugcccuggcucaguuaucacagugcugaugcugucuauucuaaagguacaguacugugauaacugaaggauggca

SEQ ID NO: 3 - DNA, 20 bp, Mus musculus
tacagtactgtgataactga

SEQ ID NO: 4 - DNA, 21 bp, Bos Taurus
gtacagtactgtgataactga

SEQ ID NO: 5 - DNA, 24 bp, Artificial Sequence
  Synthetic
tgcagttgcttcagtacccataat

SEQ ID NO: 6 - DNA, 26 bp, Artificial Sequence
  Synthetic
atccccgtgtactttcccatcataat

SEQ ID NO: 7 - DNA, 21 bp, Artificial Sequence
  Synthetic
ttcctctttgcatggaatttg

SEQ ID NO: 8 - DNA, 20 bp, Artificial Sequence
  Synthetic
agaggagtgggggaagagtc

SEQ ID NO: 9 - DNA, 20 bp, Artificial Sequence
  Synthetic
tggacgatgtgctctatgcc

SEQ ID NO: 10 - DNA, 20 bp, Artificial Sequence
  Synthetic
ggatggtgatggtttggtag

SEQ ID NO: 11 - DNA, 22 bp, Artificial Sequence
  Synthetic
tctgtaaggcccaaagtgggta

SEQ ID NO: 12 - DNA, 25 bp, Artificial Sequence
  Synthetic
acctcagcatgacaatatgtcacaa

SEQ ID NO: 13 - DNA, 20 bp, Artificial Sequence
  Synthetic
ccgacacagacaccatcaac

SEQ ID NO: 14 - DNA, 20 bp, Artificial Sequence
  Synthetic
cttttctgcccaacttctgc

SEQ ID NO: 15 - DNA, 20 bp, Artificial Sequence
  Synthetic
ggaggagagcggtggtcaaa

SEQ ID NO: 16 - DNA, 21 bp, Artificial Sequence
  Synthetic
tgtgcagctggctcaagtcaa

SEQ ID NO: 17 - DNA, 20 bp, Artificial Sequence
  Synthetic
tgcaccaccaactgcttagc

SEQ ID NO: 18 - DNA, 21 bp, Artificial Sequence
  Synthetic
ggcatggactgtggtcatgag

SEQ ID NO: 19 - DNA, 22 bp, Artificial Sequence
  Synthetic
gtactgtgataactgaaggatg

SEQ ID NO: 20 - DNA, 22 bp, Artificial Sequence
  Synthetic
attctgcttctctttgccttgt

SEQ ID NO: 21 - DNA, 21 bp, Artificial Sequence
  Synthetic
gactgaactgtcctttttcgg

SEQ ID NO: 22 - DNA, 20 bp, Artificial Sequence
  Synthetic
cctttctcaatgtgatggca

SEQ ID NO: 23 - DNA, 20 bp, Artificial Sequence
  Synthetic
ctaatgcattgccttcagca

SEQ ID NO: 24 - DNA, 20 bp, Artificial Sequence
  Synthetic
ttagcatcttgggctcacct

SEQ ID NO: 25 - DNA, 18 bp, Artificial Sequence
  Synthetic
acctggtggcaggaacac

SEQ ID NO: 26 - DNA, 18 bp, Artificial Sequence
  Synthetic
tgaggcgctgctataccc

SEQ ID NO: 27 - DNA, 19 bp, Artificial Sequence
  Synthetic
caggcgatggcctaagact

SEQ ID NO: 28 - DNA, 20 bp, Artificial Sequence
  Synthetic
cagggtaagtctgggactgc

SEQ ID NO: 29 - DNA, 21 bp, Artificial Sequence
  Synthetic
tgaagactctggggacttttg

SEQ ID NO: 30 - DNA, 25 bp, Artificial Sequence
  Synthetic
aatatacacagaggattgcttagcc

SEQ ID NO: 31 - DNA, 20 bp, Artificial Sequence
  Synthetic
tggctggttgcatatgtagg

SEQ ID NO: 32 - DNA, 21 bp, Artificial Sequence
  Synthetic
tggtgtttctggtgtctgtca

SEQ ID NO: 33 - DNA, 22 bp, Artificial Sequence
  Synthetic
aaaacaaatgttgcaatccaaa

SEQ ID NO: 34 - DNA, 21 bp, Artificial Sequence
  Synthetic
tgcaaataacaagatgcctga

SEQ ID NO: 35 - DNA, 22 bp, Artificial Sequence
  Synthetic
actgagccattgagggtaccta

SEQ ID NO: 36 - DNA, 18 bp, Artificial Sequence
  Synthetic
ccccagaaggcaaaggat

SEQ ID NO: 37 - DNA, 21 bp, Artificial Sequence
  Synthetic
gtgagacagccaatggagaac

SEQ ID NO: 38 - DNA, 22 bp, Artificial Sequence
  Synthetic
tgttcgttaggcaacagctaca

SEQ ID NO: 39 - DNA, 20 bp, Artificial Sequence
  Synthetic
cagcctttgatggtgtgtgt

SEQ ID NO: 40 - DNA, 21 bp, Artificial Sequence
  Synthetic
tccattactggagactctggg

SEQ ID NO: 41 - DNA, 25 bp, Artificial Sequence
  Synthetic
ttagctggctctgagtatgaataac

SEQ ID NO: 42 - DNA, 20 bp, Artificial Sequence
  Synthetic
gctggaaaacccaacttctg

SEQ ID NO: 43 - DNA, 26 bp, Artificial Sequence
  Synthetic
gtgactttatgcccctttagagacaa

SEQ ID NO: 44 - DNA, 26 bp, Artificial Sequence
  Synthetic
gaagggctacaactggaactggaata

SEQ ID NO: 45 - DNA, 20 bp, Artificial Sequence
  Synthetic
attcctccaggtgggtgtgg

SEQ ID NO: 46 - DNA, 21 bp, Artificial Sequence
  Synthetic
ctaagccgctgttgccttggc

SEQ ID NO: 47 - DNA, 18 bp, Artificial Sequence
  Synthetic
tcctggcccggggcctgg

SEQ ID NO: 48 - DNA, 19 bp, Artificial Sequence
  Synthetic
ctgttccccgggctcccgc

SEQ ID NO: 49 - DNA, 19 bp, Artificial Sequence
  Synthetic
tgtcccgggatcctcttct

SEQ ID NO: 50 - DNA, 21 bp, Artificial Sequence
  Synthetic
tagagacgttggtgcggaaat

SEQ ID NO: 51 - DNA, 20 bp, Artificial Sequence
  Synthetic
tagagggtcaccgcgtctat

SEQ ID NO: 52 - DNA, 20 bp, Artificial Sequence
  Synthetic
tcacaggtgctttgcagttc

SEQ ID NO: 53 - DNA, 20 bp, Artificial Sequence
  Synthetic
gtttctgctacgctgctgct

SEQ ID NO: 54 - DNA, 20 bp, Artificial Sequence
  Synthetic
caccagctcacttaccacca

SEQ ID NO: 55 - DNA, 20 bp, Artificial Sequence
  Synthetic
tactagcggttttacgggcg

SEQ ID NO: 56 - DNA, 24 bp, Artificial Sequence
  Synthetic
tcgaacaggaggagcagagagcga

SEQ ID NO: 57 - DNA, 20 bp, Artificial Sequence
  Synthetic
ctaggagggtggaggtaggg

SEQ ID NO: 58 - DNA, 20 bp, Artificial Sequence
  Synthetic
gccccaaacaggagtaatga

SEQ ID NO: 59 - DNA, 22 bp, Artificial Sequence
  Synthetic
cagtgaggtctcagcatcagca

SEQ ID NO: 60 - DNA, 22 bp, Artificial Sequence
  Synthetic
ctggaggctatgggggtacttg

SEQ ID NO: 61 - RNA, 23 nt, Artificial Sequence
  Synthetic
gaagucgaaccucgacaugacau

SEQ ID NO: 62 - RNA, 22 nt, Artificial Sequence
  Synthetic
uacaguacugugauaacugaag

SEQ ID NO: 63 - RNA, 23 nt, Artificial Sequence
  Synthetic
uucugaauuugcaaucaugacau

SEQ ID NO: 64 - RNA, 23 nt, Artificial Sequence
  Synthetic
cuucaggaaccucgacaugacau

SEQ ID NO: 65 - RNA, 23 nt, Artificial Sequence
  Synthetic
uuguuuacauucuuuguacuguc

SEQ ID NO: 66 - RNA, 22 nt, Artificial Sequence
  Synthetic
gugaaauguuuaggaccacuag

SEQ ID NO: 67 - RNA, 23 nt, Artificial Sequence
  Synthetic
aaaauuguagcuaaacauuucau

SEQ ID NO: 68 - RNA, 23 nt, Artificial Sequence
  Synthetic
uaauacugccggguaaugaugga

SEQ ID NO: 69 - RNA, 22 nt, Artificial Sequence
  Synthetic
uaauacugccugguaaugauga

SEQ ID NO: 70 - RNA, 23 nt, Artificial Sequence
  Synthetic
ugacauaacaggaaacaguauug

SEQ ID NO: 71 - RNA, 23 nt, Artificial Sequence
  Synthetic
caacuuuauauaucacaguauuu

SEQ ID NO: 72 - RNA, 23 nt, Artificial Sequence
  Synthetic
uguagacccuuucauguacugua

SEQ ID NO: 73 - RNA, 23 nt, Artificial Sequence
  Synthetic
acccuuucauguacuguacugua

SEQ ID NO: 74 - RNA, 23 nt, Artificial Sequence
  Synthetic
guacuguacaccugauacuguaa

SEQ ID NO: 75 - RNA, 23 nt, Artificial Sequence
  Synthetic
cugauacuguaaacauacuguaa

SEQ ID NO: 76 - RNA, 23 nt, Artificial Sequence
  Synthetic
aaaggcaaccgugguuacuguau

SEQ ID NO: 77 - RNA, 23 nt, Artificial Sequence
  Synthetic
auugcaggccaaaccguacugua

SEQ ID NO: 78 - RNA, 23 nt, Artificial Sequence
  Synthetic
aauagcuauauccauguacugua

SEQ ID NO: 79 - RNA, 23 nt, Artificial Sequence
  Synthetic
gaaguucauaccuaaguacugua

SEQ ID NO: 80 - RNA, 23 nt, Artificial Sequence
  Synthetic
guuaaucucuguuauguacugua